(19) United States
(12) Patent Application Publication
(10) Pub. No.: US 2022/0110674 A1
(43) Pub. Date: Apr. 14, 2022

(54) SYSTEMS AND METHODS FOR THERAPY OF PELVIC CONDITIONS

(71) Applicant: HOLOGIC, INC., Marlborough, MA (US)

(72) Inventors: Kyle Patrick Taylor, Marlborough, MA (US); Eric S. Whitbrook, Marlborough, MA (US); John Christian Knudson, Marlborough, MA (US); Caleb Ole Vainikka, Marlborough, MA (US)

(73) Assignee: HOLOGIC, INC., Marlborough, MA (US)

(21) Appl. No.: 17/510,218

(22) Filed: Oct. 25, 2021

Related U.S. Application Data
(63) Continuation of application No. PCT/US2020/029955, filed on Apr. 24, 2020.
(60) Provisional application No. 62/839,325, filed on Apr. 26, 2019.

(51) Int. Cl. A61B 18/14; A61M 1/00

(52) U.S. Cl. CPC A61B 18/1485; A61B 2018/00577; A61M 1/7415; A61M 1/774

(57) ABSTRACT

A treatment device for treatment of overactive bladder conditions operates to suction to grasp and conform a mucosal tissue of the bladder wall to a portion of the tissue treatment device, and deliver energy to non-superficial target tissue at a substantially uniform depth from the mucosal tissue. The tissue treatment device incorporates multiple instruments for different functions needed to treat overactive bladder conditions. The tissue treatment device provides simplified interfaces and mechanisms for operating each of the multiple instruments in a controlled manner. Further, the tissue treatment device is configured to have a low and smooth profile that permits the tissue treatment device to be used without a tubular sheath.

### TECHNICAL FIELD

This document relates to treatments for pelvic conditions, such as overactive bladder conditions.

### BACKGROUND

Urinary incontinence (UI) is the involuntary leakage of urine. There are several types of urinary incontinence, including urge urinary incontinence (UUI) and stress urinary incontinence (SUI). Urge urinary incontinence is the involuntary loss of urine while suddenly feeling the need or urge to urinate. Stress urinary incontinence, typically affecting females, is the involuntary loss of urine resulting from increased abdominal pressure, such as generated by physical activity, exercising, coughing, sneezing, laughing, lifting, etc. Mixed incontinence combines attributes of SUI and UUI.

Overactive bladder (OAB) is the strong, sudden urge to urinate, with or without urinary incontinence, usually with frequency and nocturia. The urge associated with overactive bladder can be assessed using the subjective experience of the patient, with or without any objectively verifiable metric, condition, behavior, or phenomena.

OAB and UI significantly affect the quality of life and the ability of patients to maintain their lifestyle, including socializing, mobility, or independence. Further, UI is one of the most common reasons for entering long-term care facilities, such as nursing homes, and is also a significant risk factor for injury due to falls resulting from hurrying to the toilet in response to urge.

### SUMMARY

Some embodiments described herein include treatments for pelvic conditions. For example, this document describes systems and methods for treating overactive bladder conditions by ablating afferent nerves located within the wall of the bladder. The technology described herein provides therapy to non-mucosal target tissue to modulate bladder function by, for example, delivering energy to denervate selected portions of the bladder, such as afferent nerves located within or proximate to the trigone region of the bladder wall, and modulate bladder function, thereby providing relief for a sense of urge, incontinence, frequency, nocturia, bladder capacity, and/or pain. Denervation can be accomplished by delivering thermal energy (e.g., using RF energy, microwaves, or high intensity focused ultrasound) to layers of the bladder wall beneath the mucosal layer, such as within or proximate to the trigone region. Alternatively or in addition, thermal energy may be delivered to neural tissue, such as a pelvic nerve or its branches, within or proximate to the bladder wall to modulate nerve traffic to or from at least a portion of the bladder, thereby modulating bladder function.

Some embodiments of the systems and methods described herein includes a treatment device configured to suction to grasp and conform a mucosal surface of the bladder wall to a portion (e.g., a suction head) of the treatment device, and deliver energy to non-superficial target tissue at a substantially uniform depth from the mucosal surface. The treatment device incorporates multiple instruments for different functions needed to treat overactive bladder conditions. The treatment device provides simplified interfaces and mechanisms for operating each of the multiple instruments in a controlled manner. Further, the treatment device is configured to have a low and smooth profile that permits the treatment device to be used without a tubular sheath. For example, the treatment device is configured to enable the multiple instruments to be inserted into a urethra and a bladder without using a tubular sheath that would otherwise be positioned through the urethra prior to inserting instruments into the bladder. For example, as described herein, the treatment device has a suction paddle and houses an endoscope and electrodes, and is configured to permit the electrodes and/or the endoscope to be fully retracted into the suction paddle of the device when not in use. Further, the suction paddle and other parts of the treatment device are configured to have a smooth exterior without sharp edges (e.g., sharp distal-end openings of electrode channels). Because no sheath is required when using the treatment device, there is no concern about a tissue irritation by a sheath and a leakage through a sheath.

In the context of this disclosure, tissue of the female anatomy targeted for energy delivery may include one or more tissue layers of the bladder wall beneath the mucosa and extending to (but not including) the anterior vaginal wall, and are collectively referred to herein as “non-superficial tissue.” Further, in the context of this disclosure, tissue of the male anatomy targeted for energy delivery may include one or more layers of the bladder wall beneath the mucosa and extending to and including the perivesical fat layer, and in the context also is referred to as “non-superficial tissue.”

One embodiment of a tissue treatment device comprises an elongate shaft having a proximal end portion, a distal end portion, an irrigation/suction channel extending between the proximal end portion and the distal end portion, and an electrode channel extending between the proximal end portion and the distal end portion. The elongate shaft may, e.g., be shaped and sized for insertion into a human urethra. The tissue treatment device further comprises an ablation electrode (e.g., a first electrode of a pair of electrodes) slidably disposed within the electrode channel of the elongate shaft, an irrigation tube, and a suction tube. The tissue treatment device further comprises a handle assembly disposed at the proximal end portion of the elongate shaft. The handle assembly comprises a handle body at least partially receiving the suction tube and the irrigation tube, and a trigger assembly configured to selectively set the tissue treatment device in one of an irrigation mode, a suction mode, and an ablation mode.

In the irrigation mode, fluid communication between the irrigation tube and the irrigation/suction channel of the elongate shaft is allowed, while fluid communication between the suction tube and the irrigation/suction channel is prevented. In the suction mode, fluid communication between the suction tube and the irrigation/suction channel of the elongate shaft is allowed, while fluid communication between the irrigation tube and the irrigation/suction channel is prevented. In the ablation mode, the ablation electrode extends from an end of the electrode channel. In one specific implementation, when the tissue treatment device is in the ablation mode, fluid communication between the suction tube and the irrigation/suction channel of the elongate shaft is allowed, while fluid communication between the irrigation tube and the irrigation/suction channel is prevented. In this specific implementation, when the tissue treatment device is in the irrigation mode or the suction mode, the ablation electrode may be retracted within the electrode channel. In another specific implementation, the trigger assembly is configured to directly transition the tissue treatment device from the irrigation mode to the suction mode to thereby directly transition the tissue treatment device from the suction mode to the ablation mode. The trigger assembly may comprise a trigger configured to be moved along a single direction to transition the tissue treatment device from the irrigation mode to the suction mode, and then from the suction mode to the ablation mode. In another specific implementation, the trigger assembly may be configured to selectively set the tissue treatment device in one of the irrigation mode, the suction mode, the ablation mode, and a resting mode. In the resting mode, fluid communication between the irrigation tube and the axial lumen of the elongate shaft is prevented, fluid communication between the suction tube and the axial lumen of the elongated shaft is prevented, and the ablation electrode is retracted within the electrode channel.

In one specific implementation of this embodiment of the tissue treatment device, the handle assembly further comprises a fluid manifold having a chamber, an irrigation port fluidly coupled to the irrigation tube, a suction port fluidly coupled to the suction tube, and a common port fluidly coupled to the irrigation/suction channel of elongate shaft. The fluid manifold is configured to be set in one of an irrigation state during which the irrigation port is fluidly coupled to the common port via the chamber, and the suction port is not fluidly coupled to the common port via the chamber, and a suction state during which the suction port is fluidly coupled to the common port via the chamber, and the irrigation port is not fluidly coupled to the common port via the chamber. In this specific implementation, the handle assembly further comprises an electrode drive carriage to which the ablation electrode is affixed. The electrode drive carriage is configured to be set in an electrode extension state during which the ablation electrode extends from the electrode channel to place the tissue treatment device in the ablation mode, and an electrode retraction state.

In this specific implementation, the trigger assembly may comprise a trigger configured to be selectively manipulated by a user into one of an irrigation trigger position, a suction trigger position, and an ablation trigger position. The trigger assembly may further comprise a first linkage mechanically coupled between the trigger and the fluid manifold. The first linkage is configured to set the fluid manifold into the irrigation state in response to manipulation of the trigger into the irrigation trigger position, thereby placing the tissue treatment device in the irrigation mode. The first linkage is further configured to set the fluid manifold into the suction state in response to manipulation of the trigger into the suction trigger position, thereby placing the tissue treatment device in the suction mode. The trigger assembly may further comprise a second linkage mechanically coupled between the trigger and the electrode drive carriage. The second linkage is configured to set the electrode driver carriage into the electrode extension state in response to manipulation of the trigger into the ablation trigger position, thereby placing the tissue treatment device in the ablation mode.

In this specific implementation, the fluid manifold may comprise a stationary body affixed to the handle body, and a movable body that is rotatable about a pivot axis relative to the stationary body. The handle body and movable body may define the fluid manifold chamber therebetween. The stationary body may comprise each of the irrigation port, the suction port, and the common port, and the movable body may comprise an irrigation stopper and a suction stopper. The first linkage may be affixed to and configured to move the movable body relative to the stationary body in response to manipulation of the trigger into the irrigation trigger position in which the irrigation stopper does not cover the irrigation port while the suction stopper covers the suction port, thereby setting the fluid manifold in the irrigation state. The first linkage may be further configured to move the movable body relative to the stationary body in response to manipulation of the trigger into the suction trigger position in which the irrigation stopper covers the irrigation port while the suction stopper does not cover the suction port, thereby setting the fluid manifold in the suction state.

In this specific implementation, when the tissue treatment device is in the ablation mode, fluid communication between the suction tube and the irrigation/suction channel of the elongate shaft may be allowed, while fluid communication between the irrigation tube and the irrigation/suction channel is prevented. The first linkage may be configured to set the fluid manifold into the suction state in response to manipulation of the trigger into the ablation trigger position, thereby placing the tissue treatment device into the ablation mode. When the tissue treatment device is in the irrigation mode or the suction mode, the ablation electrode may be retracted within the electrode channel. The second linkage may be configured to set the electrode drive carriage into the electrode retraction state in response to manipulation of trigger into either of the irrigation trigger position and the suction trigger position, thereby placing the tissue treatment device into one of the irrigation mode and the suction mode. The trigger assembly may optionally be configured to selectively set the tissue treatment device in a resting mode during which fluid communication between the irrigation tube and the axial lumen of the elongate shaft is prevented, fluid communication between the suction tube and the axial lumen of the elongated shaft is prevented, and the ablation electrode is retracted within the electrode channel.

In this specific implementation, the fluid manifold may be configured to be set in one of the irrigation state, the suction state, and a no-irrigation/no suction-state. In the no-irrigation/no suction-state, the irrigation port is not fluidly coupled to the common port via the chamber, and the suction port is not fluidly coupled to the common port via the chamber. The trigger may be configured to be selectively manipulated into one of the irrigation trigger position, the suction trigger position, the ablation trigger position, and a release trigger position. The first linkage may be configured to set the fluid manifold into the no-irrigation state/no-suction state, and the second linkage may be configured to set the electrode drive carriage into the electrode retraction state, respectively, in response to manipulation of the trigger into the release trigger position to thereby place the tissue treatment device into the resting mode.

In another specific implementation, the trigger assembly comprises a trigger configured to be selectively manipulated by a user into one of an irrigation trigger position that sets the tissue treatment device into the irrigation mode, a suction trigger position that sets the tissue treatment device into the suction mode, and an ablation trigger position that sets the tissue treatment device into the ablation mode. The trigger assembly further comprises a positioning arm extending from the trigger, and a trigger track having an arm guide route within which a free end of the positioning arm is slidably engaged to guide the positioning arm to one of an irrigation location that sets the trigger in the irrigation trigger position, a suction location that sets the trigger in the suction trigger position, and an ablation location that sets the trigger in the ablation trigger position.

In this specific implementation, the arm guide route may include a recessed channel, in which case, the free end of the positioning arm may have a trigger track pin that slidably engages the recessed channel of the arm guide route. In this specific implementation, the arm guide route may have a first retention feature configured for retaining the free end of the positioning arm at the suction location, thereby maintaining the trigger in the suction trigger position, and a second retention feature configured for retaining the free end of the positioning arm at the ablation location, thereby maintaining the trigger in the ablation trigger position. The arm guide route of the trigger track may have a first trigger path in which the positioning arm moves from the irrigation location to the suction location, a second trigger path in which the positioning arm moves from the suction location to the ablation location, a first return path in which the positioning arm moves from the suction location back to the irrigation location, and a second return path in which the positioning arm moves from the ablation location to the irrigation location.

One method of uses a treatment device to treat a trigonal region of a bladder of a patient. The treatment device comprises an elongate shaft, a tissue marking element disposed on the distal end of the elongated shaft, an endoscope disposed in an endoscope channel in the elongated shaft, and a distally located electrode carried by the elongate shaft proximal to the tissue marking element.

The method comprises introducing the treatment device through a urethra and into the bladder of the patient (e.g., a patient suffering from an overactive bladder (OAB) condition), visualizing a ureter ostium in the bladder with the endoscope, marking the ureter ostium by indenting a mucosal surface of the bladder adjacent the ureter ostium with the tissue marking element to create a temporary imprint on the mucosal surface of the bladder, positioning the electrode adjacent a target site on the trigonal region of the bladder while visualizing the indented mucosal surface with the endoscope, and energizing the electrode, thereby ablating the target site on the trigonal region of the bladder (e.g., to treat the OAB condition). In one specific implementation, the treatment device is introduced through the urethral and into the bladder while the endoscope is retracted within the endoscope channel of the elongate shaft, in which case, the method may further comprise deploying the endoscope from the endoscope channel when the treatment device is in the bladder.

In one specific implementation of the method, the treatment device further comprises a suction paddle disposed at the distal end of the elongate shaft, and the distally located electrode comprises a cannula deployable from the distal end of the elongate shaft. In this case, the method further comprises applying suction by the suction paddle to grasp the mucosal surface of the bladder, deploying the cannula of the electrode from the distal end of the elongate shaft into submucosal tissue of the bladder, and energizing the cannula to thereby ablate the submucosal tissue at the target site. In this specific implementation, the tissue marking element may comprise a paddle positioner extending from the suction paddle distal to the deployed electrode, in which case, the method may further comprise visualizing the ureter ostium through the paddle position with the endoscope, and aligning the electrode relative to the visualized ureter ostium, such that the electrode is a known distance from the visualized ureter ostium when the electrode is energized. In this specific implementation, the paddle positioner may optionally include a rib aligned with a longitudinal axis of the elongated shaft, in which case, positioning the electrode at the target site may comprise aligning the vertical rib with the ureter ostium.

The method may optionally further comprise visualizing another ureter ostium in the bladder with the endoscope, marking the other ureter ostium by indenting a mucosal surface of the bladder adjacent the other ureter ostium with the tissue marking element to create a temporary imprint on the mucosal surface of the bladder, positioning the electrode adjacent the other target site on the trigonal region of the bladder while visualizing the indented mucosal surface with the endoscope, and energizing the electrode, thereby ablating the other target site on the trigonal region of the bladder.

Another embodiment of a tissue treatment device comprises an elongate shaft and a suction paddle disposed at a distal end of the elongate shaft. The suction paddle has a suction head configured to apply a suction to a surface of tissue to grasp the tissue. The suction paddle further has a heat dissipation portion arranged in the suction head. The suction head having a first thermal conductivity, and the heat dissipation portion having a second thermal conductivity greater than the first thermal conductivity. The tissue treatment device further comprises an electrode deployable from the distal end of the elongated shaft into the tissue beneath the tissue surface. The electrode is configured for generating heat and ablating the tissue beneath the tissue surface, and the heat dissipation portion is configured for dissipating heat away from the tissue surface.

In one specific implementation, the suction head has a face and a suction aperture provided on the face. The face is configured to contact the surface of the body tissue, and the heat dissipation portion is arranged opposite to the face of the suction head. In another specific implementation, the heat dissipation portion may be made as a separate plate and mounted to the suction head. In still another specific implementation, the heat dissipation portion is formed as an integral part of the suction head. In yet another specific implementation, the heat dissipation portion is composed of a metallic material, and the suction head is composed of a polymeric material. In yet another specific implementation, a tissue treatment system may comprise this tissue treatment device and an endoscope disposed in an endoscope channel in the elongated shaft, in which case, the heat dissipation portion may be transparent, thereby allowing the tissue to be visualized by the endoscope through the heat dissipation portion.

Still another embodiment of a tissue treatment device comprises an elongate shaft and suction paddle disposed at a distal end of the elongate shaft, the suction paddle having a suction head configured to apply a suction to a surface of tissue to thereby grasp tissue to be treated. The tissue treatment device further comprises a pair of electrodes configured to be deployed from a distal end portion of the elongated shaft along a plane into the tissue so that a distal portion of each electrode is positioned beneath a surface the tissue. Each electrode of the pair comprises a beveled tip with an angled surface that is oriented along the plane and is configured to limit flexing of the respective electrode out of the plane when the electrodes are deployed from the distal end of the elongated shaft into the tissue. In one specific implementation, the respective beveled tips of the electrodes are oriented to face toward one another. In another specific implementation, the respective beveled tips of the electrodes are oriented to face away from one another. In still another specific implementation, the respective beveled tips of the electrodes are oriented to face in a same direction on the plane. In yet another specific implementation, the respective electrodes each have cannula portions, in which case, the respective beveled tips of the electrodes are provided at the respective cannula portions.

One embodiment of a medical device comprises an elongate shaft having an endoscope channel configured to receive an endoscope, such that a distal tip of the endoscope can be alternately advanced from and retracted within a distal opening of the endoscope channel. In one specific implementation, the distal opening of the endoscope channel is angled relative to the longitudinal axis of the endoscope channel. The medical device further comprises an operative medical element disposed a distal end portion of the elongate shaft and configured for performing a medical procedure on tissue. The endoscope is configured to view tissue on which the medical procedure is being performed when advanced from the distal opening of the endoscope channel. The medical device further comprises a ramp carried by the distal end portion of the elongate shaft adjacent the distal opening of the endoscope channel, the ramp being distal to the distal opening of the endoscope channel. The ramp further has an endoscope support surface on which the endoscope slides when advanced from, or retracted within, the distal opening of the endoscope channel, such that a distal tip of the endoscope diverges away from a longitudinal axis of the endoscope channel, thereby increasing a frictional force imposed on the endoscope by the endoscope channel at the distal opening of the endoscope channel. In one specific implementation, the ramp has an arcuate cross-section configured to conform to an outer surface of the endoscope.

In one specific implementation, the operative element may be an electrode configured for delivering electrical energy for ablating the tissue. In this case, the medical device may optionally comprise a suction paddle disposed on the elongate shaft distal to the distal opening of the endoscope channel. The suction paddle has a suction head configured to apply a suction to a surface of tissue to grasp the tissue, and the electrode may be configured to be advanced from the distal end of the elongated shaft into the tissue beneath a surface of the tissue, and wherein the ramp is disposed on the suction head.

In another specific implementation, the medical device further comprises a handle assembly disposed at a proximal end portion of the elongate shaft. The handle assembly comprising a handle body having an opening in communication with the endoscope channel, and an endoscope carriage configured to receive and hold the endoscope. The endoscope carriage may be configured to slide relative to the handle body to control axial movement of the endoscope within the endoscope channel. The endoscope carriage may be configured to slide distally relative to the handle body in order to advance the endoscope from the distal opening of the endoscope channel, and relative to the handle body in order to retract the endoscope into the distal opening of the endoscope channel. In this specific implementation, the handle assembly may further comprise a pair of blocks, with each block of the pair having an arcuate surface. The respective accurate surfaces of the blocks may face each other to frictionally engage the endoscope. A medical treatment device may comprise the medical device and an endoscope.

Another embodiment of a medical device comprises an elongate shaft having an endoscope channel configured to slidably receive an endoscope, an operative medical element disposed at the distal end of the elongate shaft and configured for performing a medical procedure on tissue, and a handle assembly. For example, the operative element may include an electrode configured for delivering electrical energy for ablating the tissue. The handle assembly includes a handle body having an opening in communication with the endoscope channel of the elongate shaft, and an endoscope support mechanism having an endoscope carriage disposed on or within the handle body and configured to receive and hold the endoscope. The endoscope carriage is configured to slide relative to the handle body to control axial movement of the endoscope within the endoscope channel of the elongate shaft, such that the endoscope carriage slides distally relative to the handle body to advance a distal tip of the endoscope from a distal opening of the endoscope channel of the elongate shaft to view the tissue on which the medical procedure is being performed, and slides proximally relative to the handle body to retract the distal tip of the endoscope carriage into the distal opening of the endoscope channel.

In one specific implementation, the endoscope support mechanism further comprises at least one slide guide affixed within the handle body and on which the endoscope carriage slides. In another specific implementation, the handle assembly further comprises a pair of blocks, with each block of the pair having an arcuate surface. The respective accurate surfaces of the blocks face each other to frictionally engage the endoscope.

In still another specific implementation, the endoscopic carriage is configured to slide distally within the handle body to advance the distal tip of the endoscope from the distal opening of the endoscope channel. In this specific implementation, the endoscopic sliding mechanism may further comprise a locking device configured to releasably lock the endoscope carriage within the handle body in at least one axial position. Such locking device may comprise at least one detent arranged on the interior of the handle body, and a flexible arm cantilevered at the endoscope carriage. The flexible arm may have a free end with a protrusion, and the detent(s) may be configured to engage the protrusion to releasably lock the endoscope carriage within the handle body in the axial position(s). The detent(s) may comprise a plurality of axially disposed detents on the interior of the handle body, thereby allowing the endoscopic carriage to be releasably locked within the handle body in any one of a plurality of different axial positions. A proximal end of the handle body may comprise a wall, and the protrusion may be configured to engage a proximal surface of the wall in the absence of a vertical force on the cantilevered flexible arm, thereby preventing the endoscopic carriage from being slid distally within the handle body, and to disengage the proximal surface of the wall in the presence of the vertical force on the cantilevered flexible arm, thereby allowing the endoscopic carriage to be slid distally within the handle body. The protrusion may have an angled proximal surface, such that the protrusion slidably engages a distal surface of the wall of the handle body when the endoscope carriage is slid proximally within the handle body, thereby allowing the endoscopic carriage to be slid proximally out of the handle body.

Another embodiment of a tissue treatment device comprises an elongate shaft and a first electrode carried by a distal end of the elongate shaft. The tissue treatment device may optionally further comprise a second electrode carried by a distal end of the elongate shaft in a side-by-side configuration with the first electrode. The first electrode comprises an electrically conductive hollow cannula portion having a cannula wall and a cannula lumen extending from a proximal end of the hollow cannula portion to a distal end of the hollow cannula portion. The hollow cannula portion has a notch formed in the cannula wall at or proximate to the proximal end of the hollow cannula portion. The tissue treatment device further comprises a first wire composed of a first electrically conductive material. A distal end of the first wire is affixed within the notch of the hollow cannula portion, such that the first wire is in electrical contact with the hollow cannula portion. The tissue treatment device further comprise a second wire composed of a second electrically conductive material different from the first electrically conductive material of the first wire. The second wire extends through the cannula lumen from the proximal end of the hollow cannula portion to the distal end of the hollow cannula portion. A distal end of the second wire is affixed to the distal end of the hollow cannula portion, such that the second wire is in electrical contact with the hollow cannula portion.

In one specific implementation, the notch is formed entirely through the cannula wall. In another specific implementation, the distal end of the hollow cannula portion is beveled to form a lateral opening in the cannula wall, and the distal end of the second wire is exposed through the lateral opening in the cannula wall. In still another specific implementation, the hollow cannula portion is composed of stainless steel, the first electrically conductive material of the first wire is one of copper and constantan, and the second electrically conductive material of the second wire is the other of copper and constant. In yet another specific implementation, the electrode is configured to be advanced from the distal end of the elongated shaft into tissue, and retracted from the tissue into the distal end of the elongated shaft. In yet another specific implementation, the tissue treatment system further comprises a handle assembly disposed at the proximal end of the elongate shaft. The handle assembly comprises a handle body, an electrical cable at least partially contained in the handle body, and a port through which the electrical cable extends from the handle body. The electrical cable is in electrical communication with each of the first electrically conductive wire and the second electrically conductive wire.

One embodiment of a tissue treatment system comprises the tissue treatment device and an electrical ablation source to which the electrical cable is operably coupled. The electrical ablation source is configured for measuring a voltage difference between the first electrically conductive wire and the second electrically conductive wire, and for determining a temperature of tissue adjacent the distal end of the second electrically conductive wire based on the measured voltage difference. The electrical ablation source may be further configured for energizing the electrode by conveying electrical ablation energy to one of the first electrically conductive wire and the second electrically conductive wire.

The devices, system, and techniques described herein may provide one or more of the following advantages. First, the treatment device described herein is a single device that integrates multiple instruments required for treatment of pelvic conditions, such as overactive bladder conditions, and allows simple, controlled operations of the instruments. Second, the treatment device described herein provides a suction paddle having a heat dissipation portion that can effectively remove heat from an ablation zone and thus prevent damage of the mucosal tissue resulting from the heat from the ablation zone. Third, the treatment device provides beveled tips of the electrodes oriented to cause the electrodes to flex in a same plane containing the electrodes, but prevents the electrodes from randomly flexing in different directions, such as directions up and down from the same plane. This can ensure that the ablation zone can be formed uniformly every time the treatment device is used. Fourth, the treatment device includes a simple trigger assembly that enables the treatment device to switch between different phases of procedure. The trigger assembly can use a single trigger that can be selectively pulled and released to different positions that cause the treatment device to perform different operations. Fifth, the treatment device includes several features to stabilize an endoscope and move the endoscope in a controlled manner while providing a wider field of view of the endoscope. Sixth, the treatment device can be used to effectively and conveniently mark regions in the bladder to be avoided during ablation of targeted region in the bladder. Seventh, the treatment device includes several features for measuring tissue temperature at electrodes of the treatment device using a thermocouple that minimizes the profile of an electrode while maintaining the structural integrity and electrical performance of the electrode.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a plan view of an exemplary tissue treatment system constructed in accordance with one embodiment of the disclosure.

FIG. 2A is a profile view of an exemplary tissue treatment device used in the tissue treatment system of FIG. 1.

FIG. 2B is a cross-sectional view of the tissue treatment device of FIG. 2A.

FIG. 3A is a profile view of one embodiment of a suction paddle of the tissue treatment device of FIG. 2A.

FIG. 3B is a top perspective view of the suction paddle of FIG. 3A.

FIG. 3C is a bottom perspective view of the suction paddle of FIG. 3A.

FIG. 3D is another top perspective view of the surgical paddle of FIG. 3A.

FIG. 4A is a top perspective view of another embodiment of a suction paddle of the tissue treatment device of FIG. 2A.

FIG. 4B is a top perspective view of another embodiment of a suction paddle of the tissue treatment device of FIG. 2A.

FIG. 5 is a partially cut-away top view of one embodiment of a set of electrodes used in the tissue treatment device of FIG. 2A

FIG. 6A is a partially cut-way top view of one embodiment of a cannula portion and thermocouple arrangement of an electrode of FIG. 5.

FIG. 6B is a partially cut-way side view of the cannula portion and thermocouple arrangement of FIG. 6A.

FIG. 7A is a side sectional view of the tissue treatment device of FIG. 2A introduced in the female anatomy.

FIG. 7B is a side view of the surgical paddle of FIG. 3A used in the female anatomy during an ablation procedure.

FIG. 7C is a side view of the electrodes and a heat dissipation portion of the surgical paddle of FIG. 3A, during an ablation procedure.

FIG. 7D shows an angled frontal-axial sectional view of the bottom portion of the bladder after treatment by the tissue treatment device of FIG. 2A.

FIG. 8A is a side partial cross-sectional view of an exemplary handle assembly of the treatment device of FIG. 2A when operating in an irrigation mode.

FIG. 8B is a side partial cross-sectional view of the exemplary handle assembly of the treatment device of FIG. 2A when operating in a suction mode.

FIG. 8C is a side partial cross-sectional view of the exemplary handle assembly of the treatment device of FIG. 2A when operating in an ablation mode.

FIG. 8D is a close-up view of an exemplary fluid manifold of the handle assembly of FIG. 8A;

FIG. 8E is a close-up view of an exemplary electrode support mechanism of the handle assembly of FIG. 8B.

FIG. 8F is a close-up view of an exemplary trigger assembly of the handle assembly of FIG. 8B.

FIG. 8G is a perspective exploded view of the fluid manifold of FIG. 8A.

FIG. 8H is another perspective exploded view of the fluid manifold of FIG. 8A.

FIG. 9A is a profile view of the exemplary handle assembly of the treatment device of FIG. 2A.

FIG. 9B is a perspective view of an endoscope support mechanism of the treatment device of FIG. 2A.

FIG. 9C is a side cross sectional view of the handle assembly of the treatment of FIG. 2A.

FIG. 9D is another perspective view of the endoscope support mechanism of FIG. 9B.

FIG. 10 is a front partially cutaway perspective view of the handle assembly of the treatment device of FIG. 2A.

### DETAILED DESCRIPTION OF ILLUSTRATIVE EMBODIMENTS

Some embodiments described herein include a treatment system for pelvic conditions, such as overactive bladder conditions. The system can include a treatment device configured to deliver thermal energy to denervate selected portions of the bladder, such as afferent nerves located within or proximate to the trigone region of the bladder wall, and modulate bladder function. The treatment device can be configured and operated to grasp and conform a mucosal surface of the bladder wall to a suction head of the treatment device, and deliver energy to non-superficial target tissue at a substantially uniform depth from the mucosal surface. The treatment device is configured to house multiple instruments needed for different processes in the treatment, such as an irrigation mechanism, a suction mechanism, an endoscope, and electrodes, and provide simplified interfaces and mechanisms for operating each of the multiple instruments in a controlled manner. Further, the treatment device is configured to have a low and smooth profile that permits the treatment device to be used without a tubular sheath that would otherwise be positioned through a urethra prior to inserting instruments into a bladder. Because no sheath is required when using the treatment device, there is no concern about a tissue irritation by a sheath and a leakage through the sheath.

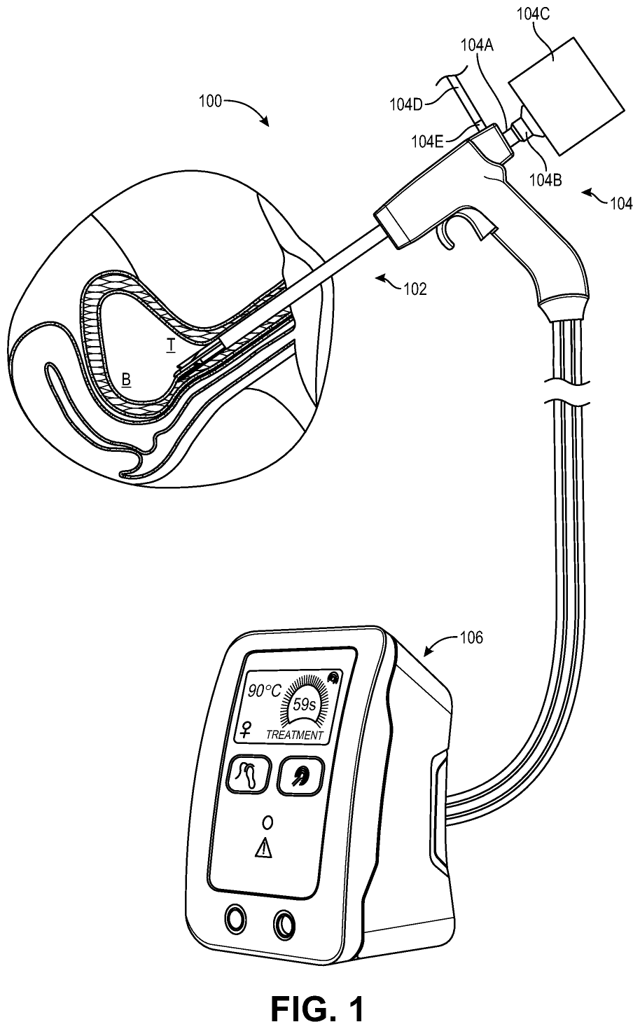

Referring to FIG. 1, an exemplary system 100 for treatment of tissue, such as a bladder B, will be described. The system 100 includes a treatment device 102, an endoscope assembly 104, and a base unit 106 connectable to the treatment device 102.

In general, the treatment device 102 may be configured as a cystoscope that facilitates visualization and therapy for treatment of overactive bladder disease (OAB). The treatment device 102 is configured to be inserted transurethrally to the bladder B, such as a trigone region T of the bladder B. As will be described in further detail below, the distal end of the treatment device 102 is used to grasp, hold, and stabilize the mucosal surface of the trigone region T when suction is applied to the treatment device 102. After the tissue of the bladder B is stabilized, the treatment device 102 is configured to advance ablation electrodes into the trigone region T for subsequent ablation of sub-mucosal tissue.

The endoscope assembly 104 includes an endoscope 104A and an eyepiece 104B connected with the endoscope 104A. The endoscope assembly 104 further includes a camera 104C that may be connected to an eyepiece 104B, and a light cable 104D that may be connected to a light adapter 140E. The endoscope assembly 104 can be of various types, such as Hopkins rod endoscopes, endoscopes having cameras at or near their tips, and any other suitable endoscopes.

The base unit 106 to which the treatment device 102 is connected is configured to control the treatment device 102 to perform various operations. For example, in the illustrated embodiment, the base unit 106 comprises a radiofrequency (RF) generator configured to provide RF ablation energy to the treatment device 102 in order to ablate nerves within the trigone region T of the bladder B. The treatment device 102 is connected to an irrigation source (e.g., an IV saline bag) and a vacuum pump. The irrigation source is configured to deliver fluid (e.g., saline) to the treatment device 102 for irrigation of the bladder B. The vacuum pump operates to create pressure drop for suction using the treatment device 102. In the illustrated embodiment, at least part of the functionalities of the vacuum pump and the irrigation source may be incorporated in the base unit 106, although it should be appreciated that the irrigation source and/or vacuum pump may reside outside of the base unit 106.

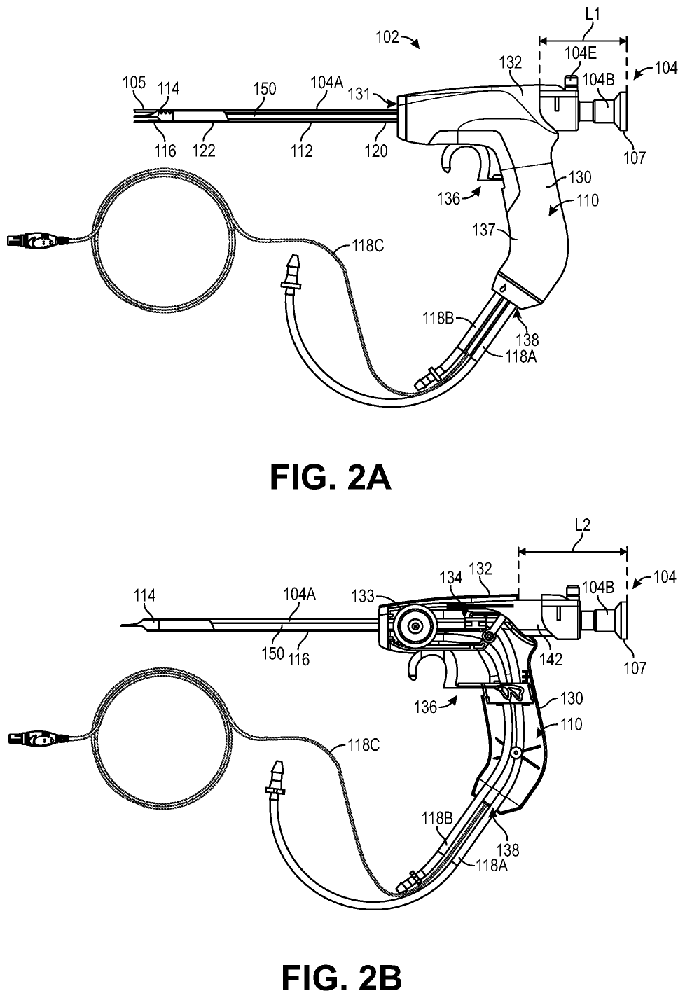

Referring to FIGS. 2A and 2B, the treatment device 102 comprises a handle assembly 110, a shaft 112, a suction paddle 114, a set of electrodes 116, one or more conduits 118 (including tubes 118A and 118B, and an electrical cable 118C).

The shaft 112 of the treatment device 102 has a proximate end 120 on which the handle assembly 110 is disposed, and a distal end 122 on which the suction paddle 114 and electrodes 116 are disposed. The shaft 112 is configured to at least partially receive the endoscope 104A within an endoscope channel (not shown), and house the electrodes 116 in electrode channels (not shown). The shaft 112 has an irrigation/suction channel (or lumen) 150 that extends from the handle body 130 of the handle assembly 110 and is partially received within the handle body 130 of the handle assembly 110. The shaft 112 may be shaped and sized for insertion into a urethra, and made of materials with flexibility desired for operations.

The handle assembly 110 is configured to movably support the endoscope assembly 104 relative to the treatment device 102, provide a grip for the user, and to control irrigation, suction, and ablation functions of the treatment device 102. The handle assembly 110 comprises a handle body 130, an endoscope support mechanism 132, a fluid manifold 133 (shown in FIG. 2B), an electrode support mechanism 134 (shown in FIG. 2B), a trigger assembly 136, and one or more ports 138 through which the tubes 118A and 118B pass out of the handle body 130 and the electrical cable 118C extends.

The handle body 130 is configured to be conveniently gripped by a hand of the user. For example, in the illustrated embodiment, the handle body 130 is configured in a handgun shape providing a grip 137. The handle body 130 is also configured to at least partially house various components of the treatment device 102. For example, the handle body 130 includes at least a portion of the endoscope support mechanism 132 and is configured to at least partially support the endoscope assembly 104. Further, the handle body 130 is configured to at least partially house the electrode support mechanism 134 and the trigger assembly 136. The handle body 130 defines the conduit ports 138 through which the conduits 118A-118C extend out from the handle body 130.

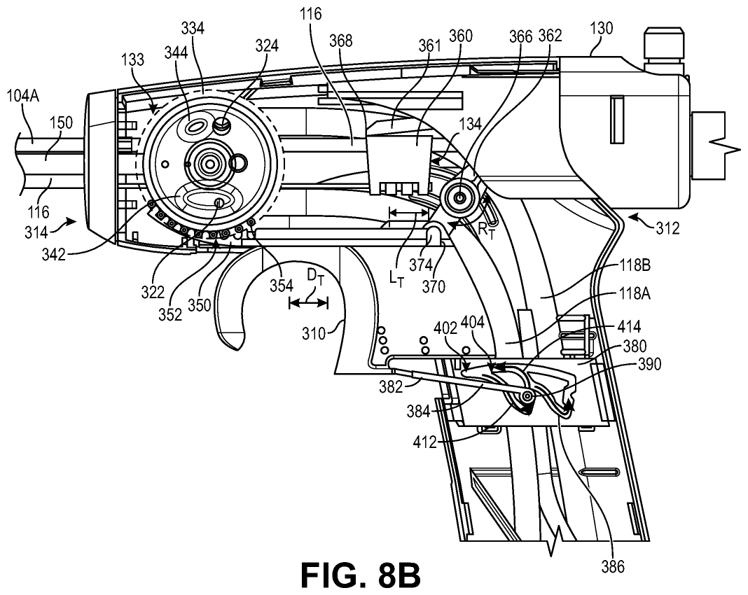

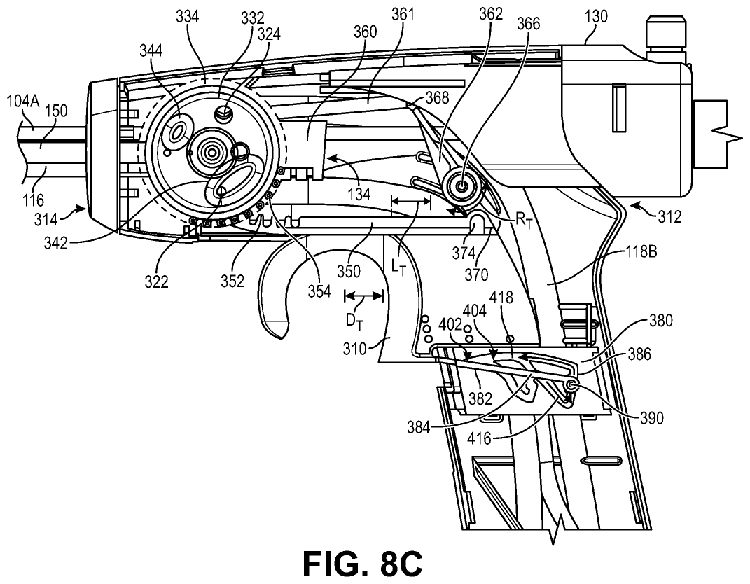

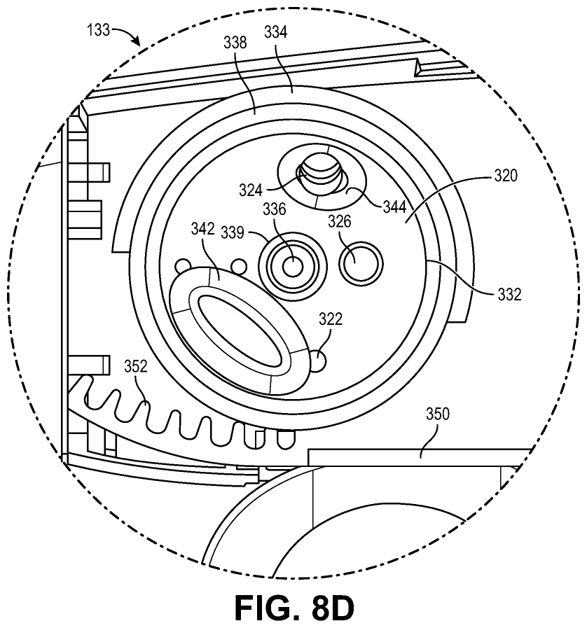

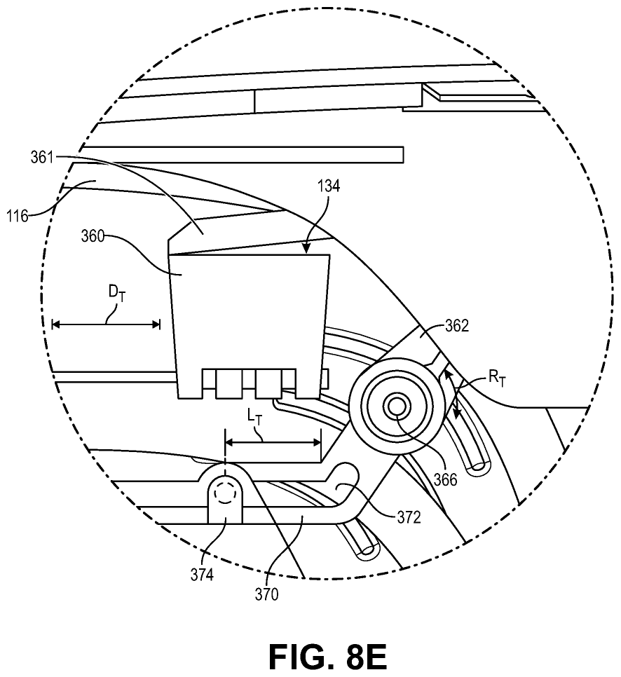

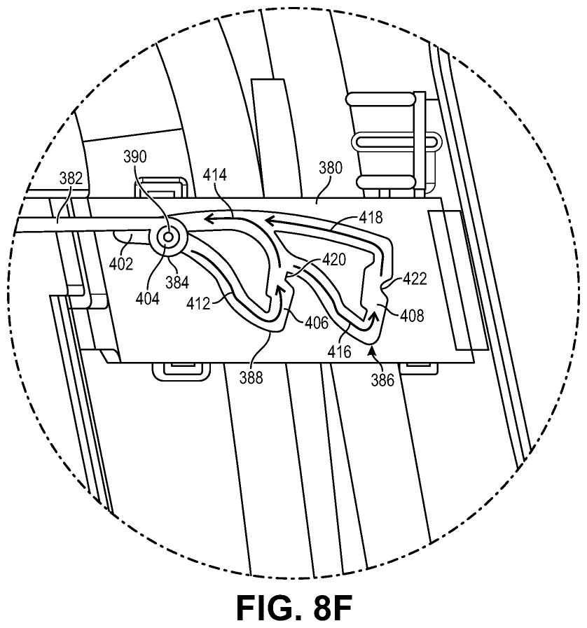

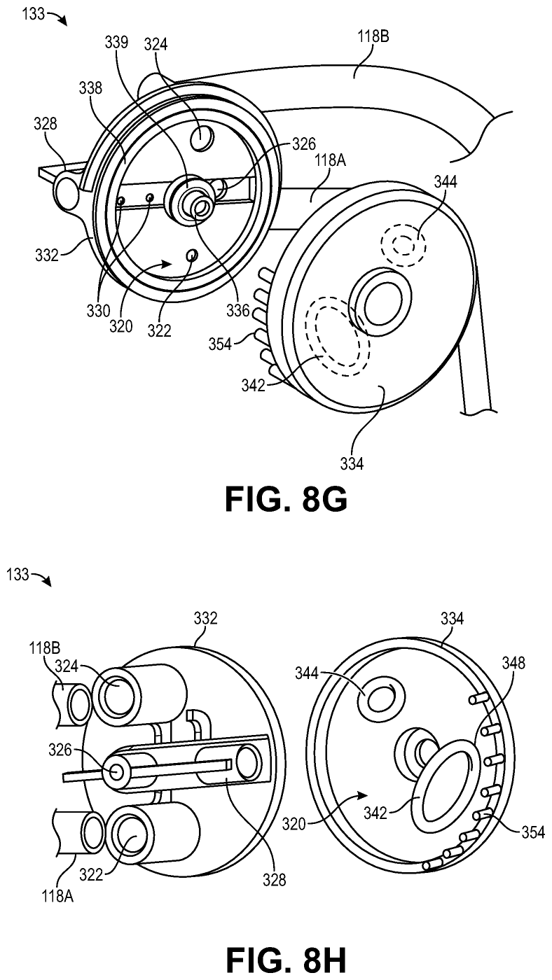

The endoscope support mechanism 132 is configured to movably receive the endoscope assembly 104, and to longitudinally move the endoscope assembly 104 relative to the handle assembly 110 between an extended position (FIG. 2A), such that a distal tip 105 of the endoscope 104A extends from the suction paddle 114 at the distal end 122 of the shaft 112, and thus is exposed, and a retracted position (FIG. 2B), such that the distal tip 105 of the endoscope 104A is retracted into the suction paddle 114 at the distal end 122 of the shaft 112, and thus is not exposed. A range of the axial movement of the endoscope assembly 104 relative to the treatment device 102 may be illustrated by a distance between a portion of the handle assembly 110 and a proximal end 107 of the endoscope assembly 104. For example, a distance L1 (FIG. 2A) between a portion of the handle assembly 110 and the proximal end 107 of the endoscope assembly 104 in the extended position is smaller than a distance L2 (FIG. 2B) between the same portion of the handle assembly 110 and the proximal end 107 of the endoscope assembly 104. The endoscope assembly 104 may be movable within a range between the distance L1 and the distance L2. Further details discussing the endoscope support mechanism 132 will be provided below with respect to FIGS. 8A-8C.

The tubes 118 (including 118A and 118B) are in selective fluid communication with the inflation/suction channel 150 within the shaft 112. The fluid manifold 133 is configured to selectively make a fluid communication between the irrigation/suction channel 150 and the irrigation tube 118A leading to the irrigation source or between the irrigation/suction channel 150 and the suction tube 118B leading to the vacuum pump. The tubes 118 can be routed within the handle body 130 from the fluid manifold 133 and extend through the conduit ports 138 at a bottom of the handle assembly 110 to the base unit 106 or otherwise an irrigation source and/or vacuum source separate from the base unit 106. The tubes 118 can be used for irrigation and/or suction purposes. For example, the treatment device 102 includes an irrigation tube 118A that may be coupled to an irrigation source, and a suction tube 118B that may be coupled to a vacuum pump. Flow control valves (not shown), such as stopcocks may be used to connect the vacuum pump and/or irrigation source to the tubes 118. In some implementations, one or more of the tubes 118 may act as a vent to the atmosphere. In some implementations, one or more of the tubes 118 may be permanently or episodically connected to a syringe, which may be used to instill or extract volumes of fluid into or out of the anatomic structure (in this case, the bladder B) in which the treatment device 102 is used.

The electrode support mechanism 134 (FIG. 2B) movably supports the set of electrodes 116. For example, the electrode support mechanism 134 is configured to enable the electrodes 116 to longitudinally (or axially) move relative to the treatment device 102 via operation of the trigger assembly 136. In particular, as illustrated in FIG. 2A, the electrodes 116 can be distally moved into an electrode extension position, so that the distal portions of the electrodes 116 extend out of the electrode channels in the shaft 112, and thus, are exposed at the distal end 122 of the shaft 112. As illustrated in FIG. 2B, the electrodes 116 can be proximally moved into an electrode retraction position, so that the distal portions of the electrodes 116 are retracted within the electrode channels of the shaft 112, and thus, are not exposed at the distal end 122 of the shaft 112.

The electrical cable 108C is in electrical communication with the electrodes 116 extending within the shaft 112. The electrical cable 108C can be routed within the handle body 130 from the electrodes 116 and extend through the conduit ports 138 at a bottom of the handle assembly 110 to the base unit 106.

The trigger assembly 136 is operably coupled to the fluid manifold 133 and the electrode support mechanism 134 to selectively set the treatment device 102 to different operational modes over the course of the procedure, and in particular, an irrigation mode, a suction mode, and an ablation mode. The trigger assembly 136 allows a single trigger to be moved to different trigger positions to conveniently switch between different modes of the treatment device 102. For example, the treatment device 102 is configured to permit the trigger assembly 136 to be selectively moved to an irrigation trigger position (in which fluid such as saline is delivered into the bladder), a suction trigger position (in which suction in the bladder B is performed), and an ablation position (in which electrodes 116 are extended into the bladder B during suction). The trigger assembly 136 is configured to select these trigger positions in a mutually exclusive manner. The trigger assembly 136 can be configured to be pulled from the irrigation trigger position to the suction trigger position, and released from the suction trigger position to the irrigation trigger position (or alternatively to a release trigger position). Further, the trigger assembly 136 can be configured to be pulled from the suction trigger position to the ablation position, and released from the ablation trigger position to the irrigation trigger position (or alternatively to the release trigger position).

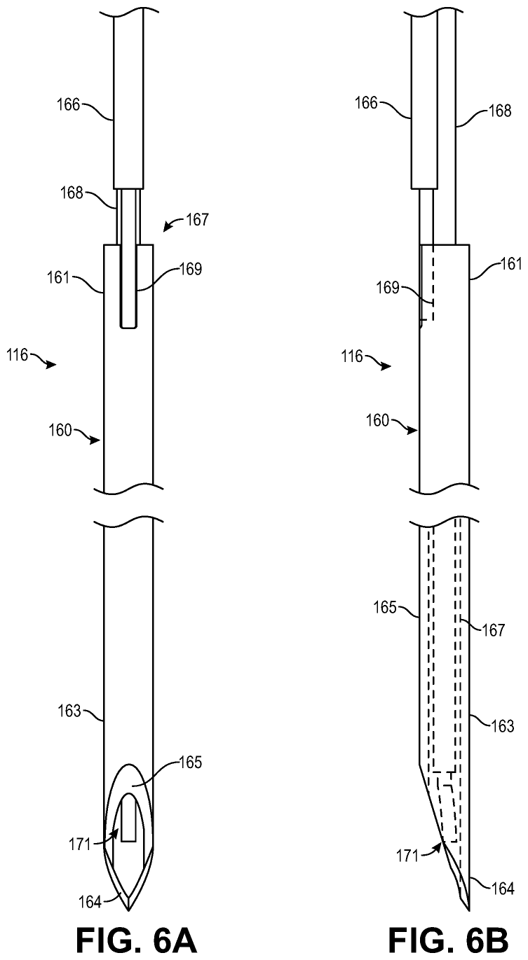

Further details discussing the fluid manifold 133, the electrode support mechanism 134, and the trigger assembly 136 will be provided in below with reference to FIGS. 6A-6C.

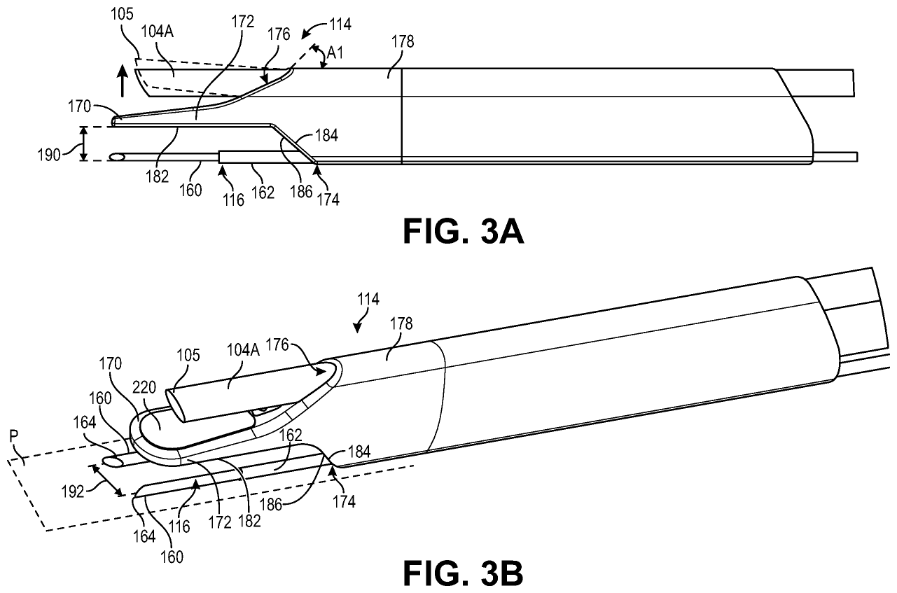

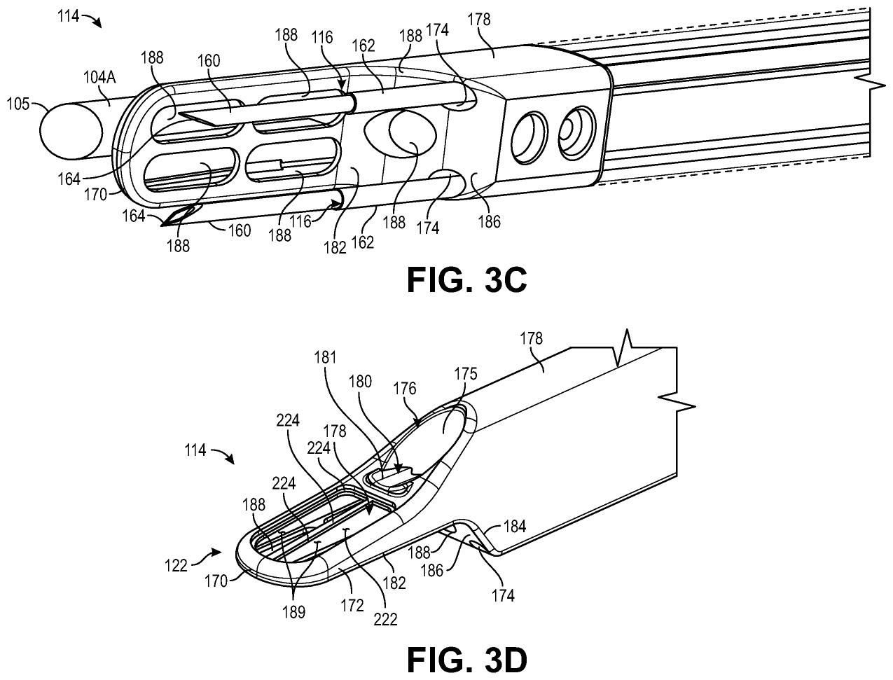

Referring now to FIGS. 3A-3D, the distal ends of the electrodes 116 include cannula portions 160, which can be energized to transfer energy into the patient. Insulation 162 surrounds the electrodes 116 except for exposed portions of the cannula portions 160, thereby limiting the effective treatment portion to the exposed portions of the cannula portions 160. Connecting wires, which connect the electrodes 116 to the base unit 106 (FIG. 1), are not illustrated in FIGS. 3A-3D. It is to be understood that any suitable electrode may be utilized with the treatment device 102. Some example types include electrodes that have a needle-shaped end or reside within a cannula, such as that manufactured by Stryker, Cosman, Neurotherm. Other example electrodes may be also contemplated, such as electrodes that are “one piece” and capable of directly penetrating tissue without an external cannula.

At least one of the electrodes 116 can be used to ablate tissue. If both of the electrodes 116 are utilized, a bi-polar current can be applied, which concentrates current in relatively planar space between the exposed portions of the electrodes 116. In some implementations, if both electrodes 116 are used in a bi-polar configuration (or if more than two electrodes are multiplexed such that they are energized in pairs), wherein the electrodes 116 are parallel to each other along their uninsulated portion, an energy deposition region is created which is uniform in a cross section along the length of the uninsulated portion, so that a treatment is uniform in thickness and width along the length of the electrodes 116.

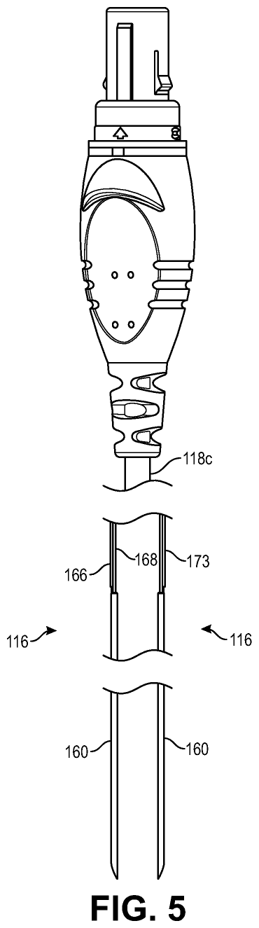

It is also preferable to use an electrode of the type that has a temperature measurement element at its tip, such as an embedded thermocouple or thermistor. The types manufactured by Stryker, Cosman, Neurotherm include this feature. One specific design of a thermocouple that can be incorporated into one of the electrodes 116 is described in detail below with respect to FIGS. 5 and 6A-6B.

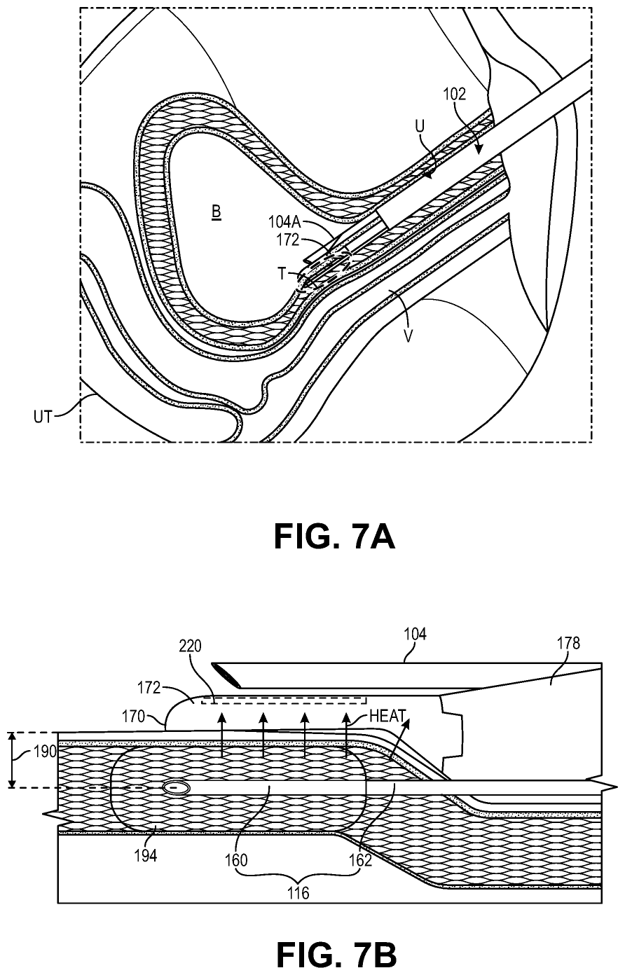

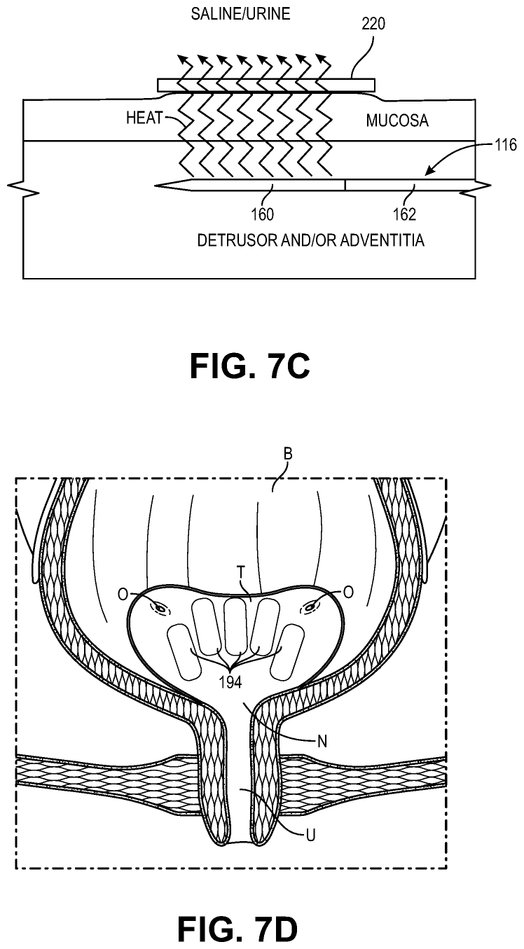

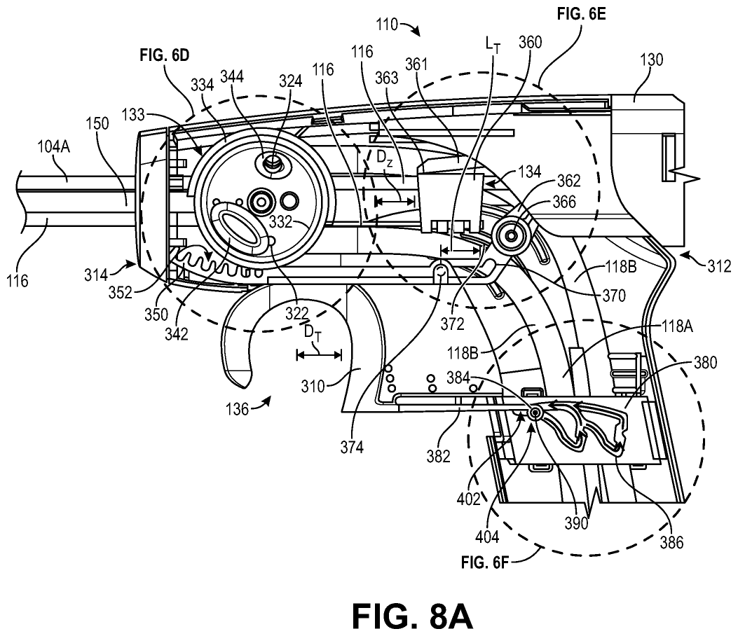

Referring to FIGS. 3B and 3C, the cannula portions 160 of the electrodes 116 have beveled tips 164 oriented to limit flexing of the electrodes 116 within a particular plane P along which the electrodes 116 are advanced into tissue, and prevent the electrodes 116 from flexing away from that plane P when the electrodes penetrate the tissue. Such orientation of the beveled tips 164 can provide a consistent, repeatable ablation zone around the electrodes 116 when the electrodes 116 penetrate into a tissue. In the illustrated implementations, the cannula portions 160 can have opposing beveled tips 164, whereby the angled surfaces of the beveled tips 164 of the cannula portions 160 face toward one another. The opposing beveled tips 164 of the cannula portions 160 can cause the cannula portions 160 to flex away from one another in a plane P that contains the cannula portions 160, as the cannula portions 160 penetrate into the tissue. This can result in a consistent ablation zone 194 (shown in FIG. 7B) around the cannula portions 160. The angled surfaces of the beveled tips 164 that face one another can limit flex of the cannula portions 160 in the plane P containing the cannula portions 160 and prevent the cannula portions 160 from flexing up and down from that plane P, which could generate inconsistent, distorted ablation zones around the cannula portions 16. Further, the opposing beveled tips 164 permit the cannula portions 160 to be bent away from one another, thereby effectively increasing the ablation zone width (e.g., the heat affected zone).

In one alternative embodiment, the cannula portions 160 can have opposing beveled tips 164. For example, the beveled tips 164 can be arranged such that the angled surfaces of the beveled tips 164 face away from one another. When the electrodes 116 penetrate into tissue, the opposite beveled tips 164 of the electrodes 116 can cause the electrodes 116 to flex toward one another in a plane that contains the electrodes 116 and prevent the electrodes 116 from flexing in opposite directions or in other directions away from the plane P.

In another alternative embodiment, the cannula portions 160 can have beveled tips 164 that face a same direction. For example, angled surfaces of the beveled tips 164 are arranged to face a same direction within a plane P that contains the electrodes. When the electrodes penetrate into a tissue, the same-direction beveled tips 164 can cause the electrodes 116 to flex in the same direction away from plane P and prevent the electrodes 116 from flexing in other directions.

The treatment device 102 can include a mechanism (not shown) configured to adjust and orient the beveled tips 164 of the electrodes 116 with respect to the treatment device 102. For example, the treatment device 102 provides an interface for manually or automatically rotating the electrodes 116 around the longitudinal axes so that they can be orientated to provide the opposing beveled tips 164, the opposite beveled tips 164, the same-direction beveled tips 164, or any other suitable orientations of the beveled tips 164. The treatment device 102 can include several features that improve control of the endoscope assembly 104, as described in further detail below.

Referring still to FIGS. 3A-3D, the suction paddle 114 of the treatment device 102 includes a suction head 172, electrode channel distal openings 174, an endoscope channel 175 with an endoscope channel distal opening 176, and a tube holder portion 178.

The suction head 172 includes a rounded, atraumatic distal end 170, a flat face 182, and a heel portion 184 with an angled (or curved) face 186. The flat face 182 and the angled face 186 define at least one suction aperture 188, as best shown in FIG. 3C. The suction apertures 188 formed on the flat face 182 lead to suction chambers 189 defined within the tube holder portion 178 (FIG. 3D) thereabove, which is in fluid communication with the irrigation/suction channel 150 (FIG. 2B) provided in the shaft 112. The suction aperture 188 formed on the angled face 186 leads to a suction chamber 189, which is in fluid communication with the irrigation/suction channel 150 provided in the shaft 112. In the illustrated embodiment, the suction head 172 includes four suction apertures 188 formed in the flat face 182 and one suction aperture 188 formed in the angled face 186. Various configurations (e.g., sizes, shapes, numbers, and/or arrangements) of suction apertures 188 may be possible. In the examples of FIGS. 3A-3D, the suction head 172 includes four longitudinally-elongated suction apertures 188 in a 2×2 arrangement in the flat face 182 and a single suction aperture 188 in the angled face 186.

The flat face 182 extends from the distal end of the suction head 172 to the heel portion 184. The shape of the suction head 172 is configured to seal itself to, and thus grasp, the mucosal surface, when a suction is applied to the suction chambers 189 (FIG. 3D). The flat face 182 establishes a seal with the soft tissue being targeted, while the angled face 186 of the heal 184 provides a gentle transition to the electrode channel distal openings 174. The heel portion 184 serves at least two functions.

The first function of the heel portion 184 is to hold the tissue being engaged by the suction apertures 188 in the angled face 186 and prevent that tissue from being pushed away from the suction head 172 when the electrodes 116 are being advanced into the tissue. The orientation of the angled face 186 assists in resisting longitudinal movement by the tissue as a result of the advancement of the electrodes 116 into the tissue.

The second function of the heel portion 184 is to provide a transition between the flat face 182 and the electrode channel distal openings 174. A vertical separation 190 (FIG. 3A) between the flat face 182 and the electrodes 116 helps define the depth at which the electrodes 112 (or cannula portions 160 thereof) will penetrate and treat the targeted tissue. This vertical separation 190 allows the electrodes 112 to engage the targeted tissue layer below the surface while avoiding or minimizing treatment of the surface layer of the bladder interior. More specifically, for bladder applications, such as ablation of portions of the trigone region T of the bladder B for treatment of overactive bladder, an example of a desired spacing is between 0.5 and 5.0 mm and preferably between 1.0 and 4.0 mm. In this manner, it is believed that the thermal treatment of the submucosal tissue is concentrated at around 0.0 to 7.0 mm depth from the bladder surface, which is where disruption of the afferent nerves is believed to be effective, while reducing thermal effects at the surface of the bladder. Greater or lesser spacing is also contemplated. A horizontal spacing 192 (FIG. 3B) between the electrodes 116 has an impact on the width of the thermal treatment zone. Some embodiments of the horizontal spacing 192 ranges from 3 to 5 mm. Other ranges are also possible.

As shown in FIG. 3A, the axes of the electrodes 116 and the suction head 172 can be parallel. For example, the electrodes 116 are at a uniform distance from the flat face 182 along the entire length of the flat face 182. Alternatively, it is anticipated that certain non-zero angles between the suction head 172 and the electrodes 116 may offer certain benefits. For example, a non-zero angle could be chosen to bias the distal portion of the electrodes 116 (and thus the therapy) to be at a different distance, either to bias the therapy to a different, preferential, depth, or to correct for differences in tissue properties tracking of the cannula portions 160 through the tissue.

As illustrated in FIGS. 2A, 3A, and 3B, the endoscope 104A passes through the endoscope channel 175 and can be advanced to extend out of the endoscope channel distal opening 176 so that the endoscope 104A has a view of the suction head 172 and/or the tissue ahead of the suction head 172. Further, as illustrated in FIG. 2B, the endoscope 104A can be retracted into the endoscope channel 175 when not in use. As best illustrated in FIG. 3A, the endoscope channel distal opening 176 can be angled relative to the endoscope channel 175. For example, the endoscope channel distal opening 176 forms an acute angle A1 relative to the longitudinal axis of the endoscope channel 175. Alternatively, the angle A1 can be an obtuse angle. Alternatively, the endoscope channel distal opening 176 is not angled relative to the endoscope channel 175.

The tube holder portion 178 is a housing that can be used to connect the various tubes/channels of the treatment device 102. The tube holder portion 178 secures the endoscope channel 175 leading to the endoscope channel distal opening 176, a channel or chamber that fluidly connects the irrigation/suction channel 150 with the suction apertures 188, and electrode channels leading to the electrode channel distal openings 174. This arrangement of the channels and chambers, with a non-circular outer shape, allows the suction paddle 114 to contain all the tubular elements in a desired arrangement, while minimizing the overall periphery dimension, thus facilitating placement of the treatment device 102 into anatomy, such as the urethra to access the bladder.

The suction paddle 114 further includes a ramp 180 configured to arrange the endoscope 104A in position that passes through the endoscope channel 175. The ramp 180 can be provided on the bottom (as viewed in FIG. 3D) of the inner surface of the endoscope channel 175 at the endoscope channel distal opening 176. The ramp 180 can have an endoscope support surface 181 on which the endoscope 104A slides when advanced or retracted. The support surface 181 can be curved to accommodate the rounded exterior of the endoscope 104A. The support surface 181 of the ramp 180 can be inclined upwards (at an upward angle) as it extends toward the distal direction. The angled support surface 181 can function to push the endoscope 104A upwards (e.g., the top center of the endoscope channel 175) as the endoscope 104A slides out of the endoscope channel 175 at the endoscope channel distal opening 176.

The angled support surface 181 can effectively provide an interference fit between the endoscope 104A and the endoscope channel 175 at the endoscope channel distal opening 176, thereby preventing the endoscope 104A from wiggling in use, and stabilizing the endoscope 104A as it extends out at the endoscope channel distal opening 176. The ramp 180 increases a frictional force imposed on the endoscope 104A by the endoscope channel 175 at the endoscope channel distal opening 176. For example, the ramp 180 may provide friction against the endoscope 104A as it moves, thereby improving a smooth, controlled movement of the endoscope relative to the treatment device 102. As depicted in FIG. 3A, the angled support surface 801 of the ramp 180 can also improve a field of view of the endoscope 104 as the ramp 180 can effectively move the endoscope 104A away (shown in phantom) from the suction head 172. The angled support surface 801 of the ramp 180 permits for the endoscope 104A to be consistently positioned in this configuration in repeated use. Although the ramp 180 is primarily described to be positioned at the bottom center of the endoscope channel 175 and at the endoscope channel distal opening 176, it is understood that other locations are possible, such as at the top center of the endoscope channel 175 and/or at the endoscope channel distal opening 176.

When the cannula portions 160 of the electrodes 116 are activated, they heat up and create an ablation zone (e.g., a heat affected zone 194, as shown in FIG. 7B). In some examples, the cannula portions 160 can be heated up to around, for example, 90° C. However, the mucosal tissue can be damaged if it is heated above, for example, 55° C. The suction paddle 114 of the treatment device 102 can be configured to keep the mucosal tissue below a predetermined temperature, such as 55° C.

It may be desirable to limit the ablation zone (e.g., the heat affected zone 194 illustrated in FIG. 7B) in the submucosal tissue, so as to spare the surface tissue and urothelium to minimize follow-up patient discomfort, risk of infection, and other benefits. One way to reduce the impact of the ablation zone 194 on the mucosal tissue is to provide a sufficient offset (e.g., the vertical separation 190) between the suction head 172 and the electrodes 116, so that the heat generated from the electrodes 116 does not significantly reach the mucosal tissue thereabove. However, this solution does not entirely eliminate the impact of the ablation zone 194 on the mucosal tissue. Further, the offset distance (e.g., the vertical separation 190) between the suction head 172 and the electrodes 116 is often limited to a particular range of distance for targeted treatments, and thus does not prevent or sufficiently reduce damage to the mucosal tissue.

The suction paddle 114 may be configured to promote heat dissipation from the ablation zone 194. In the illustrated embodiment, the suction paddle 114 further includes a heat dissipation portion 220 (best shown in FIG. 3B) configured to promote dissipation of heat from the ablation zone 194. The heat dissipation portion 220 can be positioned to correspond to the length of the cannula portions 160 (active tips) of the electrodes 116. For example, the heat dissipation portion 220 can be arranged such that, when the suction head 172 is placed onto the surface of the bladder tissue and suction is activated (as shown in FIG. 7B), the mucosal tissue is drawn into the suction head 172 through the suction apertures 188 and can contact the heat dissipation portion 220. Alternatively or in addition, the heat dissipation portion 220 is configured to be transparent or translucent so that the tissue can be viewed through the heat dissipation portion 220 by, for example, the endoscope 104A. For example, as the tissue is drawn through the suction apertures 188 and approaches the interior side of the heat dissipation portion 220, the tissue can be seen as color variations when viewed by an endoscope 104A positioned the opposite exterior side of the heat dissipation portion 220. Thus, such an optically visible configuration of the heat dissipation portion 220 can help positioning the treatment device 102 (e.g., the suction paddle 114, the electrodes 116, etc.) relative to target regions or portions of the tissue.

In some implementations, the heat dissipation portion 220 is made as a separate element and mounted to the suction head 172. For example, the heat dissipation portion 220 is configured as a plate and attached to the suction head 172. In such implementations, therefore, the heat dissipation portion 220 can also be referred to as a heat dissipation plate. The heat dissipation portion 220 can be made of one or more materials different from the material of the suction head 172 and attached to the suction head 172. For example, the heat dissipation portion 220 is made of one or more materials having a higher thermal conductivity than the material of the suction head 172, thereby increasing efficiency of heat dissipation through the heat dissipation portion 220. The heat dissipation portion 220 can be made of one or more materials of high thermal conductivity, such as copper, aluminum, stainless steel, diamond, and other suitable metallic, polymeric, or non-metallic materials. Alternatively, the heat dissipation portion 220 can be made of one or more materials which have moderate or relatively low thermal conductivity and provide other desired properties. As described herein, the suction head 172 can be made of a plastic, other suitable polymers, or other suitable electrically non-conductive materials. The heat dissipation portion 220 can be attached or inserted to the suction head 172 so as to be electrically isolated.

As illustrated in FIG. 3D, the suction head 172 can include an opening 222 on a side (e.g., a top side in FIGS. 3B and 3D) opposite to the flat face 182 having the suction apertures 188. The opening 222 can be sized to fit the heat dissipation portion 220. The opening 222 can be sealingly closed by the heat dissipation portion 220, thereby effectively forming a suction chamber in the suction paddle 114. Alternatively, the suction head 172 can include a recessed portion (without opening) configured to at least partially receive the heat dissipation portion 220 therein. The suction head 172 can include one or more ridges 224 configured to support the heat dissipation portion 220 that is at least partially received in the opening 222 of the suction head 172. The ridges 224 can be arranged around the peripheral of the opening 222, and/or arranged to transverse the opening 222.

The heat dissipation portion 220 can be mounted to the suction head 172 in various manners. In some implementations, the heat dissipation portion 220 can be formed with the suction head 172 by over-molding or insert-molding. Alternatively or in addition, the heat dissipation portion 220 can be welded, blazed, and/or soldered to the suction head 172. Alternatively or in addition, the heat dissipation portion 220 can be attached to the suction head 172 with adhesive. Alternatively or in addition, fasteners, such as screws, bolts, nuts, snaps, clamps, etc., can be used to attach the heat dissipation portion 220 to the suction head 172. For example, the heat dissipation portion 220 is at least partially inserted into the opening 222 of the suction head 172 and fixed to the suction head 172 using, for example, welding, brazing, soldering, boding with adhesive, fastening, etc. The heat dissipation portion 220 can be sealingly attached to the suction head 172.

In alternative implementations, the heat dissipation portion 220 can be made as an integral part of the suction head 172. For example, the heat dissipation portion 220 is made of the same material as the rest of the suction head 172, but has a thinner profile (a thinner wall or membrane) than the rest of the suction head. Such a thinner profile of the heat dissipation portion 220 can allow heat to effectively dissipate through the heat dissipation portion 220. The heat dissipation portion 220 can be formed in various manners, such as by milling a portion of the suction head 172, by molding the suction head 172, and by using any other suitable processes.

To protect the mucosal tissue, the heat dissipation portion 220 is arranged in the suction head 172 and above the exposed cannula portions 160 of the electrodes 116, and is configured to effectively absorb heat generated from the cannula portions 160 and dissipate the heat into the fluid (e.g., saline and/or urine) in the bladder B. As such, the heat dissipation portion 220 improves dissipation of heat being conducted through the mucosa, and thus, the mucosa can better be protected from heat-related damage.

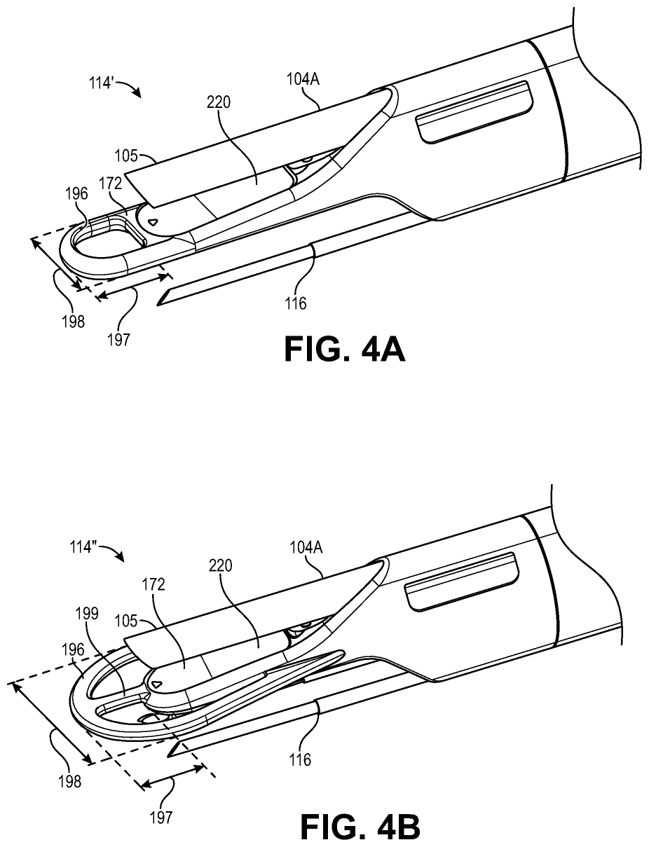

Referring to FIGS. 4A and 4B, other example suction paddles 114′, 114″, which are similar to the suction paddle 114 described in FIGS. 3A-3D, will now be described. In these examples, the suction paddles 114′, 114″ include a paddle positioner 196 arranged at the distal end of the suction head 172. The paddle positioner 196 extends from the suction head 172 and is configured to aid in the placement of the treatment device 102 relative to a desired anatomy to be treated. For example, to position the suction head 172 in a desired position relative to a uretic ostium in the bladder, the paddle positioner 196 can be visualized with the endoscope 104A and visually lined up with a ureter ostium. This can help assure that the electrodes 116 that are extended into the tissue will end up a desired distance from the ureter ostium so that, when the electrodes 116 are activated, they do not adversely affect the tissue of the ureter or its ostium. In addition or alternatively, the paddle positioner 196 can be used to indent the mucosal surface to create an imprint on the mucosal surface, which stays visible for some time (e.g., a few seconds).

The paddle positioner 196 can be configured in various shapes, such as one or more hoops, loops, plates, pointers, cross hairs, circles, wedges, or any other shapes useful in providing a visual guide. The paddle positioner 196 can be configured to have curved distal profiles. The paddle positioner 196 can be made integrally with at least a part of the suction head 172. Alternatively, the paddle positioner 196 is made separately and attached to the suction head 172 using, for example, welding, brazing, soldering, and/or bonding with adhesive. The paddle positioner 196 can be made of stainless steel. Other materials can be used, including polymeric and elastomeric materials. For example, the hoop may be made of a flexible material to ensure that it is atraumatic.

The paddle positioner 196 is configured to have a length 197 sized to enable the extended electrodes 116 to be close, but not at, the ureter ostium. The paddle positioner 196 can have a width 198 of various sizes. In FIG. 4A, the width 198 of the paddle positioner 196 is identical or similar to the width of the suction head 172. In FIG. 4B, the width 198 of the paddle positioner 196 is larger than the width of the suction head 172. The paddle positioner 196 in FIG. 4B includes a rib 199, which can be used to position the treatment device 102 with respect to the center the ostium. For example, the rib 199 is arranged to be centered on the suction head 172 (and/or the treatment device 102), so that the suction head 172 (and/or the treatment device 102) is aligned with the ostium when the rib 199 is aligned with the ostium.

It is understood that the paddle positioner 196 can be added to the other embodiments of the treatment device 102, such as the suction paddle 114 in FIGS. 3A-3D.

In some implementations, the treatment device 102 is configured such that the distal tip of the endoscope 104A does not extend above the paddle positioner 196 when the endoscope 104A is fully extended out, so that the view of the paddle positioner 196 is not obstructed by the position of the endoscope 104A. The distal tip of the endoscope 104A can be angled to face forward and downward (e.g., 45 degree), so that the endoscope 104A can have a field of view that covers both ahead and downward. Thus, the angled distal tip of the endoscope 104A allows viewing both the suction head 172 and the paddle positioner 196 together while the distal tip of the endoscope 104A is positioned above the suction head 172 and does not extend above the paddle positioner 196.

As briefly discussed above, a thermocouple for measuring the temperature of the tissue that is currently being ablated may be incorporated into one of the electrodes 116. Typically, the wires of a thermocouple are joined directly together to create a dissimilar metal interface for providing voltages therebetween that are correlated to changes in temperature to which the thermocouple is exposed. It is desirable that the temperature at the distal tips of electrodes (where the highest temperature is) be determined. To locate the dissimilar metal interface (i.e. the thermocouple) at the distal tip of the electrode, a typical embodiment would either have to route the wires of the thermocouple through a lumen of the electrode, which requires the cross-sectional profile electrode to be large enough to house the two thermocouple wires, or embed the thermocouple wires along the entire wall of the electrode, thereby degrading the structural and electrical performance of the electrode.

With reference to FIGS. 5 and 6A-6B, one embodiment of a thermocouple allows the profile of the electrodes 116 to be minimized. In particular, as illustrated in FIG. 5, the pair of electrodes 116 are shown, and as illustrated in FIGS. 6A-6B, the electrode 116 including the thermocouple arrangement is shown. The cannula portion 160 of one of the electrodes 116 is hollow, including a cannula wall 165 and a cannula lumen 167 extending from the proximal end 161 of the cannula portion 160 to the distal end 163 of the cannula portion 160. The cannula portion 160 may be composed of a suitable electrically conductive material, such as stainless steel. The electrode 116 further comprises a first thermocouple wire 166 composed of a first electrically conductive material (e.g., copper) and a second thermocouple wire 168 composed of a second different electrically conductive material (e.g., constantan).

The distal end of the first thermocouple wire 166 is embedded in the cannula wall 165 at the proximal end 161 of the cannula portion 160. In the illustrated embodiment, the cannula portion 160 has a notch 169 formed in the cannula wall 165 at the proximal end 161 of the cannula portion 160, with the distal end of the first thermocouple wire 166 being affixed within the notch 169, e.g., via welding. In the illustrated embodiment, the notch 169 is formed entirely through the cannula wall 165. However, because the notch 169 resides only at the proximal end 161 of the cannula portion 160, any degradation in the structural integrity or electrical performance of the electrode 116 is minimized. Alternatively, the notch 169 may not extend entirely through the cannula wall 165 as long as the notch 169 is deep enough to fully accommodate the distal end of the first thermocouple wire 166, such that the wire 166 does not protrude circumferentially from the cannula portion 160.

The second thermocouple wire 168 extends through the cannula lumen 167 from the proximal end 161 of the hollow cannula 165 to the distal end 163 of the hollow cannula 165, with the distal end of the second thermocouple 168 being affixed to the inside surface of the cannula wall 165 at the distal end 163 of the cannula portion 160, e.g., via welding. The beveled tip 164 of the cannula portion 160 forms a lateral opening 171, such that the distal end of the second thermocouple wire 168 is exposed through the lateral opening 171. In this manner, the second thermocouple wire 168 will be in contact with the tissue from which temperature measurements will be acquired.

In the illustrated embodiment, the thermocouple wires 166, 168 of the electrode 116, except for their distal ends, are electrically insulated to prevent shorting of the thermocouple wires 168. The thermocouple wires 166, 168 of the electrode 116, along with an electrically conductive wire 173 of the other electrode 116, extend through the respective electrode channel (not shown) of the shaft 112 back to the handle assembly 110, where they are affixed to the electrode support mechanism 134 to facilitate placement of the electrodes 116 between the electrode extension position and electrode retraction position, as briefly discussed above, and as will be discussed in further detail below.

The base unit 106 is configured for measuring the voltage difference (potential) between the thermocouple wires 166, 168, and determining the temperature of tissue (preferably during an ablation procedure) based on the measured voltage difference. In the illustrated embodiment, the base unit 106 may convey electrical ablation energy to one of the thermocouple wires 166, 168 (e.g., the first thermocouple wire 166), thereby energizing the cannula portion 160 of the electrode 116 to ablate tissue. In this embodiment, one of the thermocouple wires 166, 168 both provides the means for measuring temperature and means for ablating the tissue adjacent the cannula portion 160 of the electrode 116.

It should be appreciated that the cannula portion 160 of the electrode 116 becomes an electrical component in the thermocouple circuit between the junction joint formed between the copper material of the first thermocouple wire 166 and the cannula wall 165 at the proximal end 161 of the cannula portion 160 and the junction joint formed between the constantan material of the second thermocouple wire 168 and the cannula wall 165 at the distal end 163 of the cannula portion 160. The thermocouple can be calibrated, e.g., by taking data measurements (temperature versus voltage) and generating a temperature-voltage calibration curve that characterizes the thermocouple.

Referring to FIGS. 7A-7D, an example procedure of a body tissue treatment is described, which uses the system 100, including the treatment device 102. In the illustrated example, the treatment device 102 is primarily described as being used to treat bladder conditions such as overactive bladder (OAB). It is understood that the embodiments of the system 100 described herein may be useful to perform various other procedures and methods.

FIG. 7A is a side sectional view showing a female anatomy, which includes the bladder B, the uterus UT, the vagina V, and the urethra U. The trigone region T is shown in a dashed region. FIG. 7D shows an angled frontal-axial sectional view of the bottom portion of the bladder B, including the trigone region T, the ureteric ostia O, the bladder neck N, and the urethra U. While use of system 100 is described in connection with the female anatomy, the system 100 is contemplated for use in the male anatomy as well. Some design alterations may be used, including lengthening portions of the treatment device 102, and/or making the treatment device 102 more flexible and/or deflectable.

The treatment device 102 may be first inserted into the urethra U and into the bladder B, as shown in FIG. 7A. The treatment device 102 and the endoscope 104A may be inserted directly into the urethra U without using a sheath. Alternatively, the treatment device 102 may be placed through a prior positioned tubular sheath (not shown). When the treatment device 102 is being inserted into the bladder B, the treatment device 102 can be controlled to maintain the endoscope 104A and the electrodes 116, so that the distal tip 105 of the endoscope 104A and the cannula portions 160 of the electrodes 116 do not extend out from the endoscope channel distal opening 176 and electrode channel distal openings 174, thereby allowing smooth insertion of the treatment device 102 into the bladder B. When inserted into the bladder B, the treatment device 102 can be manipulated to place the suction head 172 onto the surface of the bladder tissue. Such placement can be performed by advancing the distal tip 105 of the endoscope 104A from the endoscope channel distal opening 176 while viewing the suction head 172 and its surrounding area through the endoscope 104A.

If the target tissue is the trigonal region T of the bladder B, it may be desirable to initially identify one of the ureteric ostia O (shown in FIG. 7D). The ureteric ostia O may be marked ahead of time by placement of a guide wire, a suture loop, or may be just visualized by the endoscope 104A during the placement of the treatment device 102, with care to avoid placement of the treatment device 102 at or too close the ureteric ostia O. Alternatively or in addition, the paddle positioner 196 of the suction paddles 114′, 114″ (FIGS. 4A and 4B) can be used to mark the ureteric ostia O, such as by indenting the mucosa to create a temporary imprint on the mucosa, which stays visible for a limited amount of time (e.g., a few seconds) and disappears. The suction head 172 may be positioned at a target ablation site on the trigonal region T of the bladder B while visualizing the indented mucosal surface with the endoscope 104A, such that the cannula portions 160 may be advanced from the electrode channel distal openings 174 into the tissue at the target site. In a preferred method, the tip of the suction head 172 is placed just medial to the ureteric ostia O. In another embodiment, the suction head 172 is placed just inferior to the ureteric ostia O. In both cases, the ureter itself is protected, since as the ureter travels lateral and superior away from the visible ureteric ostia O, placements medial and inferior avoid the obscured ureter.

When the suction head 172 is placed at the target site of the bladder B, an irrigation operation can be performed using the treatment device 102. For example, the treatment device 102 is activated (e.g., by moving the trigger assembly 136 (FIGS. 2A and 2B) to an irrigation trigger position, described in further detail below) to deliver fluid (e.g., saline) into the bladder B. The irrigation operation can be performed with or without the endoscope 104A extending out from the suction paddle 114. In addition or alternatively, the irrigation operation can be performed before the suction head 172 is placed onto the target surface of the bladder B. In some implementations, the irrigation operation can be optional.

While the suction head 172 is placed onto the surface of the bladder B, a suction operation can be performed. For example, the treatment device 102 is operated (e.g., by moving the trigger assembly 136 (FIGS. 2A and 2B) to a suction trigger position, as described in further detail below) to activate suction at the suction head 172, causing the mucosal tissue of the bladder B to come into intimate contact with, and thus to be grasped by, the face of the suction head 172, as shown in FIG. 7B. Though not shown, the mucosal tissue may actually protrude within the suction apertures 188 on the suction head 172 and contact the heat dissipation portion 220.

The suction engages and holds secure the tissue relative to the treatment device 172. Once the tissue is firmly secured to the suction head 172, the endoscope 104A is preferably withdrawn to a point where the scope tip is closer to the proximal end of the suction head 172. This facilitates observation of the electrode advancement step. The endoscope 104A may also be retracted just after the atraumatic distal end 170 on the suction head 172 is placed near the ostium, but before the suction is applied to the tissue. In some implementations, use of movement stabilization devices connected to the handle assembly 110 (FIGS. 2A and 2B) are contemplated, for example, which may be beneficial to stabilize the position of the treatment device 102 after the suction is activated and the tissue engaged with the suction head 172.

As illustrated in FIG. 7B, the electrodes 116 (or the cannula portions 160 thereof) are now advanced from the electrode channel distal openings 174 into the tissue below the surface as prescribed by the offset distance of the electrodes 116 to the flat face 182 of the suction head 172. Advancement of the electrodes 116 can be performed while the suction is in operation. For example, the treatment device 102 is operated (e.g., by moving the trigger assembly 136 (FIGS. 2A and 2B) to an ablation trigger position, as described in further detail below) to advance the electrodes 116 into the tissue while performing the suction operation. Once the electrodes 116 are arranged in position, the cannula portions 160 of the electrodes 116 may be energized by passage of electric current between them, which heats and ablates the tissue surrounding them and in between them, resulting in the heat affected zone 194. The heat affected zone 194 is preferably concentrated at a depth in the tissue, which is promoted by the dissipation of the heat from the mucosal tissue by the heat dissipation portion 220. It is believed that afferent nerves emanating from the bladder trigone region T may be ablated to lessen the sensory signals driving overactive bladder B.

Preferably the electric current is in the radio-frequency range, and preferably it is delivered in a bi-polar fashion between the two electrodes 116. Such bi-polar electrodes 116 include thermocouples for closed-loop control, in which the temperature is controlled to be limited to a set point in the software. However, it is also contemplated that the two electrodes 116 could form a mono-pole, and electric current could pass from them to a grounding pad, in a monopolar fashion. It is also contemplated, that a single electrode 116 be utilized as a monopolar current source. Multipolar configurations are also contemplated, either as single electrodes 116 that are multipolar along their lengths or as multiple electrodes 116 (3 or more) that are multiplexed or powered such that they operate in bi-polar modes, but possible in shifting patters. i.e., three electrodes 116 that form 2 bipolar pairs (middle electrode 116 is the common).

Once the treatment of the target location is performed, the suction may be released by operating the treatment device 102 (e.g., by releasing the trigger assembly 136 (FIGS. 2A and 2B) from the ablation trigger position to the irrigation trigger position (or alternatively, a release trigger position) and/or by venting the suction head 172 to atmosphere. The treatment device 102 may then be positioned in a different target location, and another ablation step may be performed, and repeated as many times as may be necessary to treat the bladder B. As illustrated in FIG. 7D, repeated ablation steps may create a pattern of heat affected zones 194. A number of different ablation patterns may be considered for treatment of the bladder B.

Referring now to FIGS. 8A-8C, the fluid manifold 133, electrode support mechanism 134, and trigger assembly 136 will be described in further detail.

As briefly discussed above, the fluid manifold 133 is configured to selectively make a fluid communication between the irrigation/suction channel 150 and the irrigation tube 118A leading to the irrigation source or between the irrigation/suction channel 150 of the shaft 112 and the suction tube 118B leading to the vacuum pump.

To this end, and with specific reference to FIGS. 8A, 8D, and 8G-8H, the fluid manifold 133 has a chamber 320 (best shown in FIGS. 8D and 8G-8H), an irrigation port 322 fluidly coupled to the irrigation tube 118A, a suction port 324 fluidly coupled to the suction tube 118B, and a common port 326 (FIG. 6H) fluidly coupled to the irrigation/suction channel 150 of shaft 112. The fluid manifold 133 is configured to be selectively set in an irrigation state, whereby the irrigation port 322 is fluidly coupled to the common port 326 (i.e., fluid is allowed to flow between the irrigation tube 118A and the irrigation/suction channel 150 of the shaft 112) via the chamber 320, and the suction port 324 is not fluidly coupled to the common port 326 via the chamber 320 (i.e., flow is prevented between the suction tube 118B and the irrigation/suction channel 150 of the shaft 112, and a suction state, whereby the suction port 324 is fluidly coupled to the common port 326 via the chamber 320 (i.e., flow is allowed between the suction tube 118B and the irrigation/suction channel 150 of the shaft 112), and the irrigation port 322 is not fluidly coupled to the common port 326 via the chamber 320 (i.e., flow is prevented between the irrigation tube 118A and the irrigation/suction channel 150 of the shaft 112).

The fluid manifold 133 includes a stationary body 332 and a movable body 334, which cooperate together to form the chamber 320 therebetween. The stationary body 332 can be fixed to the handle body 130, and the movable body 334 is movably engaged with the stationary body 332. For example, the movable body 334 is rotatable around a pivot axis 336 and relative to the stationary body 332. The stationary body 332 of the fluid manifold 133 includes the suction port 324, the irrigation port 322, and the common port 326. As best shown in FIG. 8H, the irrigation port 322 is aligned with an open end of the irrigation tube 118A and makes a fluid communication between the fluid manifold 133 and the irrigation tube 118A. The suction port 324 is aligned with an open end of the suction tube 118B and makes a fluid communication between the fluid manifold 133 and the suction tube 118B. The common port 326 is aligned with an open end of the irrigation/suction channel 150 (not shown) and makes a fluid communication between the fluid manifold 133 and the irrigation/suction channel 150. As best shown in FIG. 8G, the fluid manifold 133 further comprises a channel mount structure 328 that mounts the irrigation/suction channel 150 to the stationary body 332 of the fluid manifold 133. The channel mount structure 328 includes one or more adhesive bond ports 330 configured to bond the irrigation/suction channel 150 to the fluid manifold 133 with adhesive. The movable body 334 can be sealingly engaged with the stationary body 332, such as using an outer O-ring 338 and an inner O-ring 339, so that fluid (e.g., irrigation fluid from the irrigation tube 118A, and/or urine from the bladder) does not leak through the interface between the stationary body 332 and the movable body 334 of the fluid manifold 133.

The movable body 334 of the fluid manifold 133 includes an irrigation stopper 342 and a suction stopper 344. The irrigation stopper 342 is configured to close the irrigation port 322 of the stationary body 332 and stop the fluid communication between the fluid manifold 133 and the irrigation tube 118A. The suction stopper 344 is configured to close the suction port 324 of the stationary body 332 and stop the fluid communication between the fluid manifold 133 and the suction tube 118B. The irrigation stopper 342 can take the form of a sealing element, such as an O-ring, configured to be disposed around the irrigation port 322 and sealingly shut off the flow through the irrigation port 322. The suction stopper 344 can likewise take the form of a sealing element, such as an O-ring, configured to be disposed around the suction port 324 and sealingly shut off the flow through the suction port 324.

The irrigation stopper 342 and the suction stopper 344 are arranged on the movable body 334, such that they are selectively aligned with the suction port 324 and the irrigation port 322 of the stationary body 332 depending on the position of the movable body 334 relative to the stationary body 332. For example, the movable body 334 can be rotated relative to the stationary body 332 and arranged between an irrigation manifold position (FIG. 8A), a first suction manifold position (FIG. 8B), and a second suction manifold position (FIG. 8C).

When the movable body 334 is in the irrigation manifold position (FIG. 8A), the suction stopper 344 fully covers the suction port 324 and blocks flow through the suction port 324, while the irrigation stopper 342 does not cover (or partially covers) the irrigation port 322 to allow flow through the irrigation port 322, thereby setting the fluid manifold 133 in the irrigation state. When the movable body 334 is in the first suction manifold position (FIG. 8B), the suction stopper 344 does not cover (or partially covers) the suction port 324 to allow flow through the suction port 324, while the irrigation stopper 342 fully covers the irrigation port 322 and blocks flow through the irrigation port 322, thereby setting the fluid manifold 133 in the suction state. When the movable body 334 is in a second suction manifold position (FIG. 8C), the suction stopper 344 does not cover (or partially covers) the suction port 324 to allow flow through the suction port 324, while the irrigation stopper 342 fully covers the irrigation port 322 and block flow through the irrigation port 322, thereby again setting the fluid manifold in the suction state.

In an optional embodiment, the fluid manifold 133 is configured to be further set in a no-flow state, whereby the irrigation port 322 is not fluidly coupled to the common port 326 (i.e., flow is prevented between the irrigation tube 118A and the irrigation/suction channel 150 of the shaft 112) via the chamber 320, and the suction port 324 is not fluidly coupled to the common port 326 via the chamber 320 (i.e., flow is prevented between the suction tube 118B and the irrigation/suction channel 150 of the shaft 112. In this case, the movable body 334 can be rotated relative to the stationary body 332 and arranged between a no-flow irrigation manifold position, the irrigation manifold position (FIG. 8A), the first suction manifold position (FIG. 8B), and the second suction manifold position (FIG. 8C). The movable body 334 of the fluid manifold 133 may include two irrigation stoppers (not shown) configured to close the irrigation port 322 of the stationary body 332 and stop the fluid communication between the fluid manifold 133 and the suction tube 118B at two separate times (i.e., when the fluid manifold 133 is in the no-flow state and in the suction state). The suction stopper 344 may be made larger in the direction of the circumference of the movable body 334, such that it continually closes the suction port 324 as the fluid manifold 133 transitions between the no-flow state and the irrigation state.

In some implementations, the handle body 130 includes a window (not shown) arranged to permit a user to view the status of flow through tubes, channels, and paths (e.g., the tubes 118, the irrigation/suction channel 150, and the fluid manifold 133) within the handle body 130. For example, a window made of a transparent or translucent material is provided to a portion of the handle body 130 (e.g., close to the fluid manifold 133 that connects the tubes 118 and the irrigation/suction channel 150) through which one or more of the tubes 118, the irrigation/suction channel 150, and the fluid manifold 133 can be viewed while the treatment device 102 is operated.

Referring now to FIGS. 8A-8C and 8E, the electrode support mechanism 134 is configured to be selectively set in an electrode retraction state (FIGS. 8A and 8B), whereby cannula portions 160 of the electrodes 116 are retracted within the electrode channel distal openings 174 (shown in FIGS. 2B and 3D), such that the cannula portions 160 are not exposed, and an electrode extension state (FIG. 8C), whereby the cannula portions 160 of the electrodes 116 extend out of the electrode channel distal openings 174, such that the cannula portions 160 are exposed (shown in FIGS. 2A and 3A-3C). As best shown in FIG. 8E, the electrode support mechanism 134 includes an electrode drive carriage 360 supported in the handle body 130 and configured to mount the electrodes 116. The electrode drive carriage 360 is supported in the handle body 130 and configured to mount the electrodes 116. The electrode drive carriage 360 is fixedly coupled with the electrodes 116 at its bottom end. The electrode drive carriage 360 is movable along an electrode drive direction DE to advance and retract the electrodes 116.

The trigger assembly 136 is configured to coordinate the states of the fluid manifold 133 and the states of the electrode support mechanism 134, such that, (1) when the treatment device is in the optional rest mode, the fluid manifold 133 is in the no-flow state (i.e., the movable body 334 relative to the stationary body 332 is in the no-flow manifold position), such that neither the irrigation tube 118A nor the suction tube 118B are in fluid communication with the irrigation/suction channel 150 of the shaft 112, and the electrode support mechanism 134 is in the electrode retraction state, such that the cannula portions cannula portions 160 of the electrodes 116 are retracted within the electrode channel distal openings 174; (2) when the treatment device 102 is in the irrigation mode (FIG. 8A), the fluid manifold 133 is in the irrigation state (i.e., the movable body 334 relative to the stationary body 332 is in the irrigation manifold position), such that the irrigation tube 118A is in fluid communication with the irrigation/suction channel 150 of the shaft 112, while the suction tube 188B is not in fluid communication with the irrigation/suction channel 150 of the shaft 112, and the electrode support mechanism 134 is in the electrode retraction state, such that the cannula portions cannula portions 160 of the electrodes 116 are retracted within the electrode channel distal openings 174; (3) when the treatment device 102 is in the suction mode (FIG. 8B), the fluid manifold 133 is in the suction state (i.e., the movable body 334 relative to the stationary body 332 is in the first manifold suction position), such that the irrigation tube 118A is not in fluid communication with the irrigation/suction channel 150 of the shaft 112, while the suction tube 188B is in fluid communication with the irrigation/suction channel 150 of the shaft 112, and the electrode support mechanism 134 is in the electrode retraction state, such that the cannula portions cannula portions 160 of the electrodes 116 are retracted within the electrode channel distal openings 174; and (4) when the treatment device 102 is in the ablation mode (FIG. 8B), the fluid manifold 133 is in the suction state (i.e., the movable body 334 relative to the stationary body 332 is in the second manifold suction position), such that the irrigation tube 118A is not in fluid communication with the irrigation/suction channel 150 of the shaft 112, while the suction tube 188B is in fluid communication with the irrigation/suction channel 150 of the shaft 112, and the electrode support mechanism 134 is in the electrode extension state, such that the cannula portions cannula portions 160 of the electrodes 116 extend from the electrode channel distal openings 174.

To this end, the trigger assembly 136 includes a trigger 310 movably supported by the handle body 130. The trigger 310 is movable along a trigger direction DT. For example, the trigger 310 can be pulled in a proximal direction to progressively move into an optional trigger release position, an irrigation trigger position, a suction trigger position, and an ablation trigger position as described in further detail, and released in a distal direction to return to the suction trigger position (or optionally the release trigger position). The trigger 310 can be biased toward the distal direction to the suction trigger position (or optionally the release trigger position) using a biasing member (e.g., spring element, flexible member, etc.) to let the trigger 310 to return to another trigger by simply releasing the trigger 310.

The trigger assembly 136 includes a first linkage (and in the illustrated embodiment, a trigger rack 350 with a linear gear 352, and a manifold gear 354 (shown in FIGS. 8B-8C and 8D)) mechanically coupled between the trigger 310 and the movable body 334 of the fluid manifold 133, such that the movable body 334 can be actuated by movement of the trigger 310. In the illustrated embodiment, the linkage comprises the trigger rack 350 connected to the trigger 310, whereas the fluid manifold gear 354 is affixed around the movable body 334 of the fluid manifold 133 and engaged with the linear gear 352 of the trigger rack 350 (as in a rack and pinion configuration).

As the trigger 310 moves along the trigger direction DT by pulling or releasing the trigger 310, the trigger rack 350 can move in the same direction, the movable body 334 rotates as the linear gear 352 linear moves. In particular, the movable body 334 rotates counterclockwise as the trigger 310 is pulled, and rotates clockwise as the trigger 310 is released. As the trigger 310 moves from the optional trigger release position to the irrigation trigger position (FIG. 8A), the trigger rack 350 moves in the proximal direction, thereby rotating the movable body 334 counterclockwise, via interaction between the linear gear 352 and the manifold gear 354, from the no-flow manifold position to the irrigation manifold position. As the trigger 310 moves from the irrigation trigger position (FIG. 8A) to the suction trigger position (FIG. 8B), the trigger rack 350 moves further in the proximal direction, thereby further rotating the movable body 334 counterclockwise, via interaction between the linear gear 352 and the manifold gear 354, from the irrigation manifold position to the first manifold suction position. As the trigger 310 moves from the suction trigger position (FIG. 8B) to the ablation trigger position (FIG. 8C), the trigger rack 350 moves even further in the proximal direction, thereby further rotating the movable body 334 counterclockwise, via interaction between the linear gear 352 and the manifold gear 354, from the first suction manifold position to the second manifold suction position. When the trigger 310 is released, the trigger rack 350 moves in the distal direction, thereby rotating the movable body 334 clockwise from the second suction manifold position (FIG. 8C) back to the first suction manifold position (FIG. 8B), then from the first suction manifold position (FIG. 8B) back to the irrigation manifold position (FIG. 8A), and then optionally from the irrigation manifold position (FIG. 8A) to the no-flow manifold position.

The trigger assembly 136 includes a second linkage (and in the illustrated embodiment, a pivot arm 362 and a hinge arm 361 (shown in FIGS. 8A-8C and 8E) mechanically coupled between the trigger 310 and the electrode drive carriage 360. The pivot arm 362 is supported in the handle body 130 and pivots around a first pivot axis 366, and the hinge arm 361 is pivotally coupled with the electrode drive carriage 360 at a second pivot axis 368, so that the pivoting action of the pivot arm 362 translates into the movement of the electrode drive carriage 360 along the electrode drive direction DT. The hinge arm 361 is mechanically coupled between the electrode drive carriage 360 and the pivot arm 362. The hinge arm 361 is pivotally connected to the electrode drive carriage 360 at one end (at a third pivot axis 363 in FIG. 8A), and pivotally connected to the pivot arm 362 at the other end (at the second pivot axis 368 in FIG. 8C). The hinge arm 361 is configured to smoothly translate the pivoting action of the pivot arm 362 around the first pivot axis 366 (shown as rotational direction RT) into the linear movement of the electrode drive carriage 360 along the electrode drive direction DT.

The pivot arm 362 includes a trigger slide portion 370 arranged opposite to the second pivot axis 368 about the first pivot axis 366 of the pivot arm 362. The trigger slide portion 370 is configured to enable the trigger 310 to move (e.g., slide) along the trigger direction DT within a predetermined distance LT while leaving the pivot arm 362 stationary. In the illustrated example, the trigger slide portion 370 provides a longitudinal slot 372 extending in the trigger direction DT and having a length identical or similar to the distance LT. The trigger 310 is movably coupled to the trigger slide portion 370 by a slide pin 374 that passes through the slot 372 of the trigger slide portion 370.

Therefore, as the trigger 310 moves from the optional release trigger position or from the irrigation trigger position (FIG. 8A) to the suction trigger position (FIG. 8B), the slide pin 374 of the trigger 310 can slide along the length of the slot 372 of the trigger slide portion 370, thereby making the pivot arm 362 remain stationary until the slide pin 374 of the trigger 310 contacts an end 373 of the slot 372 of the trigger slide portion 370. As the trigger 310 moves from the suction trigger position (FIG. 8B) to the ablation trigger position (FIG. 8C), the slide pin 374 of the trigger 310 remains abutted against the end 373 of the slot 372 to push back the trigger slide portion 370 toward the proximal end 312 of the handle body 130, thereby causing the pivot arm 362 to rotate around the first pivot axis 366 (counterclockwise in FIG. 8B). The rotation of the pivot arm 362 translates into the movement of the electrode drive carriage 360 toward the distal end 314 of the handle body 130 along the electrode drive direction DE, thereby causing the electrodes 116 to advance toward the distal end 122 of the treatment device 102.

As best shown in FIGS. 8A-8C and 8F, the trigger assembly 136 includes a trigger track 380 configured to position the trigger 310. The trigger track 380 is arranged within the handle body 130 and movably engages with the trigger 310. For example, the trigger assembly 136 includes a positioning arm 382 extending from the trigger 310 and configured to flex relative to the trigger 310. The trigger track 380 includes an arm guide route 386 configured to guide the positioning arm 382. The arm guide route 386 can be provided in various configurations. In the illustrated example, the arm guide route 386 includes a recessed channel 388 that is routed in a predetermined manner. Other configurations, such as a pass-through slot or opening, can be used to make the arm guide route 386.

The positioning arm 382 has a free end 384 is slidably engaged with the arm guide route 386. For example, a trigger track pin 390 is provided at the free end 384 of the positioning arm 382, and configured to engage with and guided by the recessed channel 388 of the arm guide route 386. As the free end 384 of the positioning arm 382 is guided by the recessed channel 388 of the arm guide route 386, the positioning arm 382 can be flexed relative to the trigger 310 to accommodate the routing of the recessed channel 388 of the arm guide route 386.

The arm guide route 386 can be configured to provide a plurality of different positions of the trigger 310, which cause different functions of the treatment device 102. In the illustrated embodiment, the arm guide route 386 is configured to arrange the trigger 310 in four positions, such as an optional release trigger position, an irrigation trigger position (FIG. 8A), a suction trigger position (FIG. 8B), and an ablation trigger position (FIG. 8C). To this end, as illustrated in FIGS. 8A-8C, the arm guide route 386 includes a release location 402, an irrigation location, a suction location 406, and an ablation location 408, a first trigger path 412, a first return path 414, a second trigger path 416, and a second return path 418.

When the trigger 310 is in the optional release trigger position, the free end 384 of the positioning arm 382 is arranged in or adjacent the release location 402 of the arm guide route 386. In this trigger position, the suction port 324 and the irrigation port 322 in the fluid manifold 133 are closed, and the electrodes 116 are retracted, so that the treatment device 102 is in a rest mode.

When the trigger 310 is pulled from the optional release trigger position and moved to an irrigation trigger position, the free end 384 of the positioning arm 382 is arranged in or adjacent the irrigation location 404 of the arm guide route 386, as illustrated FIG. 8A. The irrigation trigger position of the trigger 310 also moves the movable body 334 in the irrigation manifold position in which the suction port 324 is closed and the irrigation port 322 is open, thereby triggering an irrigation operation of the treatment device 102. In some implementations, the arm guide route 386 does not provide a retention feature (e.g., notch, indent, etc.) configured to retain the free end 384 (e.g., the trigger track pin 390) of the positioning arm 382 at the irrigation location 404. Thus, a user needs to pull the trigger 310 and maintain it in the irrigation trigger position (e.g., by using the index figure) to continue the irrigation operation with the treatment device 102. Alternatively, the arm guide route 386 includes a retention feature (e.g., notch, indent, etc.) configured to receive and retain the free end 384 (e.g., the trigger track pin 390) of the positioning arm 382 at the irrigation location 404 without requiring a user's continuous pull action against the trigger 310.

When the trigger 310 is further pulled from the irrigation trigger position, the trigger 310 can move to the suction trigger position in which the free end 384 of the positioning arm 382 is arranged in or adjacent the suction location 406 as illustrated in FIG. 8B. From the irrigation location 404 to the suction location 406, the free end 384 of the positioning arm 382 can move along the first trigger path 412 of the arm guide route 386. The suction trigger position of the trigger 310 also moves the movable body 334 to the first suction manifold position in which the suction port 324 is open and the irrigation port 322 is closed, but the electrodes 116 remain retracted, thereby triggering a suction operation of the treatment device 102 without the electrodes 116 advanced. The arm guide route 386 can include a retention feature 420 (e.g., notch, indent, etc.) configured to releasably retain the free end 384 (e.g., the trigger track pin 390) of the positioning arm 382 without requiring a user's continuous pull action against the trigger 310. Alternatively, the arm guide route 386 does not include such a retention feature so that the user needs to keep pulling the trigger 310 in the suction trigger position.

In some implementations, the trigger 310 can be released from the suction trigger position back to the irrigation trigger position or back to the release trigger position. The release action can be permitted by the arm guide route 386 that guides the free end 384 of the positioning arm 382 through the first return path 414. For example, when the free end 384 is retained by the retention feature 420 in the suction location 406, the trigger 310 can be slightly pulled and released so that the free end 384 is removed from the retention feature 420 and follows the first return path 414 that is curved back to the irrigation location or further to the release location 402 (past the irrigation location) of the arm guide route 386. In other implementations, the arm guide route 386 does not provide the first return path 414, or the first return path 414 is blocked in the arm guide route 386, so that the trigger 310 is prevented from returning from the suction trigger position (e.g., the suction operation with no ablation operation) to the irrigation trigger position (e.g., the irrigation operation) or to the release trigger position (e.g., no operation).

When the trigger 310 is further pulled from the suction trigger position, the trigger 310 can move to the ablation trigger position in which the free end 384 of the positioning arm 382 is arranged in or adjacent the ablation location 408 as illustrated in FIG. 8C. From the suction location 406 to the ablation location 408, the free end 384 of the positioning arm 382 can move along the second trigger path 416 of the arm guide route 386. The ablation trigger position of the trigger 310 also arranges the movable body 334 to the second manifold position in which the suction port 324 is open and the irrigation port 322 is closed, and the electrodes 116 are advanced, thereby allowing an ablation operation of the treatment device 102 with the suction operation. The arm guide route 386 can include a retention feature 422 (e.g., notch, indent, etc.) configured to releasably retain the free end 384 (e.g., the trigger track pin 390) of the positioning arm 382 without requiring a user's continuous pull action against the trigger 310. Alternatively, the arm guide route 386 does not include such a retention feature so that the user needs to maintain the pulling action against the trigger 310 in the ablation trigger position.

Then, the trigger 310 can be released from the ablation trigger position back to the irrigation trigger position or back to the release trigger position. The release action can be permitted by the arm guide route 386 that guides the free end 384 of the positioning arm 382 through the second return path 418. For example, when the free end 384 is retained by the retention feature 422 in the ablation location 408, the trigger 310 can be slightly pulled and released so that the free end 384 is removed from the retention feature 422 and follows the second return path 418 that is routed back to the irrigation location or to the release location 402 (past the irrigation location).

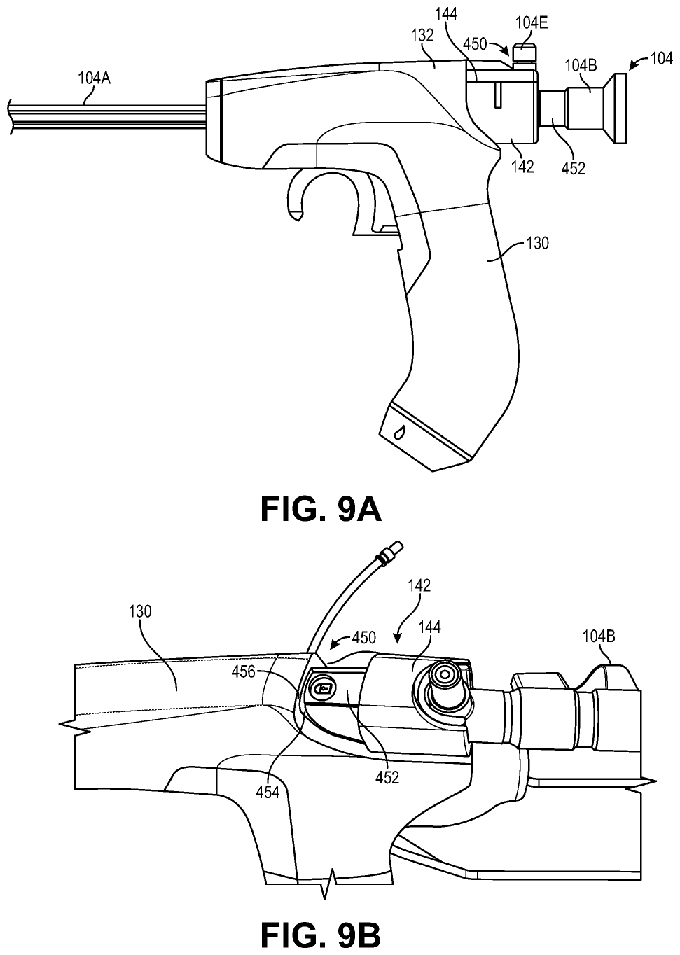

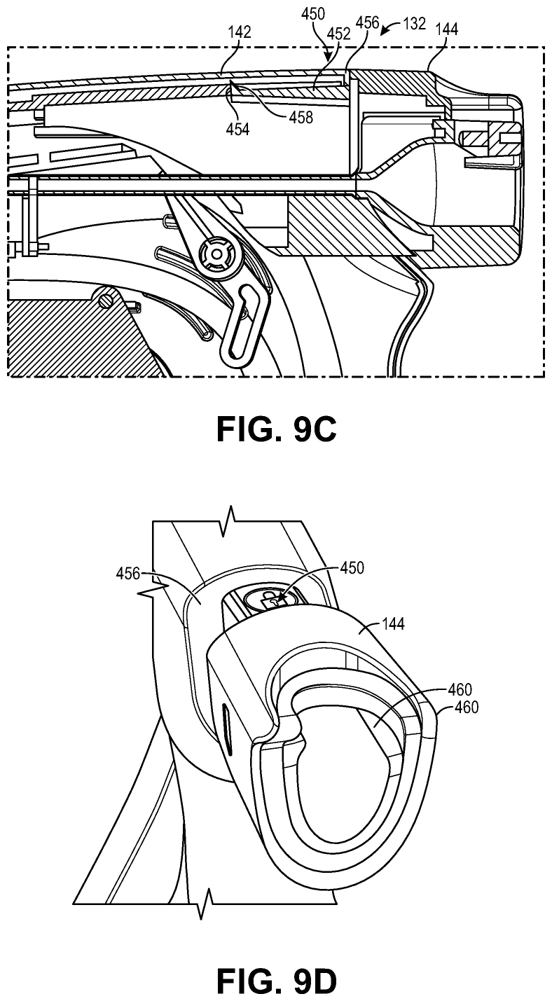

Referring to FIGS. 2B and 9A-9D, the endoscope support mechanism 132 will now be described in further detail. The endoscope support mechanism 132 includes an endoscope displacement mechanism 142 configured to receive and control longitudinal (or axial) placement of the endoscope assembly 104 relative to the handle body 130 between an extended position (FIG. 2A) and a retracted position (FIG. 2B).

In the illustrated embodiment, the endoscope displacement mechanism 142 comprises an endoscope carriage 144 that moves relative to the handle body 130 and slide guides (not shown) affixed within the handle body 130. The endoscope carriage 144 is configured to releasably hold the endoscope assembly 104, and is slidably supported by the handle body 130 along the slide guides 144. Alternatively or in addition, other manual and/or automatic configurations are possible, such as dials, rack-and-pinion mechanisms, trigger mechanisms, rocker switch configurations, worm drives, gears, stepper motors, and the like. As shown in FIG. 2A, in the extended position, the endoscope carriage 144 is moved (e.g., pushed) in a linear direction toward the distal end 122 shaft 112, such that a distal tip 105 of the endoscope assembly 104 extends from the suction paddle 114 and thus is exposed. As shown in FIG. 2B, in the retracted position, the endoscope carriage 144 is moved (e.g., pulled) away from the distal end 122 of the shaft 112, such that the distal tip 105 of the endoscope assembly 104 is retracted into the suction paddle 114 at the distal end 122 of the shaft 112.

The endoscope support mechanism 132 further includes a locking device 450 configured to limit the range of axial movement of the endoscope carriage 144 relative to the handle body 130 of the handle assembly 110, e.g., by releasably locking the endoscope carriage 144 relative to the handle body 130 in one or more predetermined positions. Further, the locking device 450 can prevent accidental movement of the endoscope carriage 144 relative to the handle body 130, such as accidental pushing when inserting an endoscope assembly 104 into the endoscope carriage 144.

In the illustrated implementations of FIGS. 9A-9D, the locking device 450 includes a flexible arm 452 that is cantilevered at the endoscope carriage 144. The flexible arm 452 can include a protrusion 454 at a free end of the flexible arm 452 and is sized to be engageable with a wall 456 that defines an opening of the handle body 130 for slidably receiving at least a portion of the endoscope carriage 144. As illustrated in FIGS. 9A and 9B, when the endoscope carriage 144 is retracted from the handle body 130 of the handle assembly 110, the protrusion 454 is engaged with the wall 456 of the handle body 130 and stops the endoscope carriage 144 from sliding into the handle body 130 until the flexible arm 452 is flexed (pushed down) until the protrusion 454 disengages from the wall 456 of the handle body 130. To enable the endoscope carriage 144 to slide into the handle body 130, the flexible arm 452 is flexed downwards so that the protrusion 454 is disengaged and released from the wall 456 of the handle body 130. When the protrusion 454 is flexed and positioned lower than the wall 456, the endoscope carriage 144 can be slid into the handle body 130 of the handle assembly 110, as illustrated in FIG. 9C.

The protrusion 454 can have an angled surface 458 that faces the inner side of the wall 456. The angled surface 458 is configured to be engaged with the inner side of the wall 456 as the endoscope carriage 144 is slid out from the handle body 130. When engaged with the inner side of the wall 456, the angled surface 458 of the protrusion 454 causes the flexible arm 452 to flex against the wall 456, thereby permitting the protrusion 454 of the flexible arm 452 to slide out from the handle body 130 as the endoscope carriage 144 is slid away from the handle body 130.

In addition or alternatively, the treatment device 102 includes one or more detents provided in the interior of the handle body 130 and configured to engage with the protrusion 454 of the flexible arm 452 as the endoscope carriage 144 moves relative to the handle body 130. For example, a row of multiple detents may be arranged along a longitudinal direction (e.g., a direction along which the endoscope carriage 144 moves) and each configured to engage with the protrusion 454 as the endoscope carriage 144 moves. Movement and arrangement of the endoscope carriage 144 relative to the handle body 130 can be guided by engagement between the protrusion 454 and each of the detents. For example, the endoscope carriage 144 can be arranged in different positions depending on which detent engages the protrusion 454.

Referring to FIG. 9D, the endoscope carriage 144 can include endoscope holding arms 460 configured to receive and hold a portion of the endoscope assembly 104 within a pocket defined in the carriage 144. In the illustrated implementations, the arms 460 are disposed to be opposing and configured to be flexed as the portion of the endoscope assembly 104 is received into (pushed in), or taken out from (pulled out from), the pocket in the carriage 144.

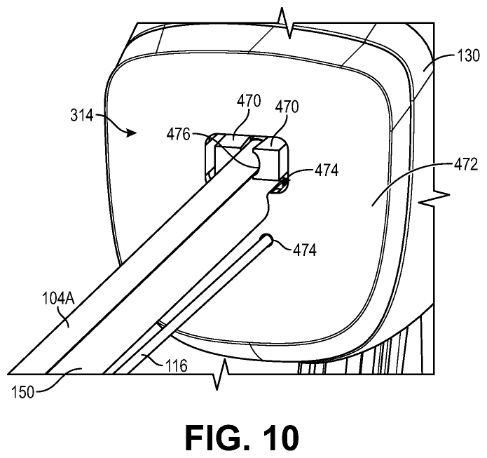

Referring to FIG. 10, the treatment device 102 includes endoscope tabs 470 configured to engage the endoscope assembly 104 for stabilization and smooth control. The endoscope tabs 470 can be arranged in the handle body 130 and adjacent the distal end 314 of the handle body 130 from which the endoscope 104A extends out. In some embodiments, the handle body 130 includes a cover 472 mounted to the distal end 314 and having one or more openings 474 through which the endoscope 104A, the irrigation/suction channel 150, and the electrodes 116 pass. The endoscope tabs 470 can be arranged adjacent the openings 474.

The endoscope tabs 470 can take the form of two blocks that face one another. Each of the blocks 470 has an arcuate surface 476 that accommodates the exterior of the endoscope 104A. For example, the arcuate surface 476 can be shaped to be rounded to correspond to the rounded exterior of the endoscope 104A. The endoscope tabs 470 can be disposed at a distance that permits for either or both of the opposing surfaces 706 of the endoscope tabs 470 to engage with the endoscope 104A, thereby generating friction against the endoscope 104A moving through the openings 474 of the handle body 130. Such friction can allow smooth, controlled movement of the endoscope 104A through the handle body 130. Further, the endoscope tabs 470 can prevent inadvertent sliding of the endoscope 104A relative to the handle body 130. The endoscope tabs 470 can be made of one or more materials, such as plastic or other suitable materials that can generate desired friction against the moving endoscope 104A.

The endoscope tabs 470 can be fixedly arranged in the handle body 130 of the treatment device 102. Alternatively, the endoscope tabs 470 can be movably arranged in the handle body 130 such that a distance between the endoscope tabs 470 is adjustable to generate different amounts of friction against the endoscope, or release the endoscope from the endoscope tabs. The endoscope tabs 470 allow the treatment device 102 to support an endoscope assembly 104 using the frictional engagement between the endoscope tabs 470 and an endoscope 104A, and require no or little frictional engagement with a holster (e.g., the endoscope sliding mechanism 142) of the endoscope assembly 104. Frictional engagement with a holster (e.g., the endoscope sliding mechanism 142) of an endoscope assembly 104 can cause separation between movement of the endoscope 104A and movement of the holster, thereby resulting in inaccurate control of the endoscope 104A through the holster.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

1-65. (canceled)

66. A tissue treatment device (102), comprising:
an elongate shaft (112) having a proximal end portion, a distal end portion, an irrigation/suction channel (150) extending between the proximal end portion and the distal end portion, and an electrode channel extending between the proximal end portion and the distal end portion;
an ablation electrode (116) slidably disposed within the electrode channel of the elongate shaft;
an irrigation tube (118A);
a suction tube (118B); and
a handle assembly (110) disposed at the proximal end portion of the elongate shaft, the handle assembly comprising
a handle body (130) at least partially receiving the suction tube and the irrigation tube; and
a trigger assembly (136) configured to selectively set the tissue treatment device in one of an irrigation mode, a suction mode, and an ablation mode, wherein in the irrigation mode, fluid communication between the irrigation tube (118A) and the irrigation/suction channel (150) of the elongate shaft (112) is allowed, while fluid communication between the suction tube (118B) and the irrigation/suction channel (150) is prevented, in the suction mode, fluid communication between the suction tube (118B) and the irrigation/suction channel (150) of the elongate shaft (112) is allowed, while fluid communication between the irrigation tube (118A) and the irrigation/suction channel (150) is prevented, and in the ablation mode, the ablation electrode (116) extends from an end of the electrode channel; characterized in that the handle assembly further comprises
a fluid manifold (133), the fluid manifold having a chamber (320), an irrigation port (322) fluidly coupled to the irrigation tube (118A), a suction port (324) fluidly coupled to the suction tube (118B), and a common port (326) fluidly coupled to the irrigation/suction channel (150) of elongate shaft (112), wherein the fluid manifold (133) is configured to be set in one of an irrigation state, wherein the irrigation port (322) is fluidly coupled to the common port (326) via the chamber (320), and the suction port (324) is not fluidly coupled to the common port (326) via the chamber (320), and a suction state, wherein the suction port (324) is fluidly coupled to the common port (326) via the chamber (320), and the irrigation port (322) is not fluidly coupled to the common port (326) via the chamber (320), and
an electrode drive carriage (360) to which the ablation electrode (116) is affixed, wherein the electrode drive carriage (360) is configured to be set in an electrode extension state, wherein the ablation electrode (116) extends from the electrode channel to place the tissue treatment device in the ablation mode, and an electrode retraction state.

67. The tissue treatment device (102) of claim 66, wherein, when the tissue treatment device (102) is in the ablation mode, fluid communication between the suction tube (118B) and the irrigation/suction channel (150) of the elongate shaft (112) is allowed, while fluid communication between the irrigation tube (118A) and the irrigation/suction channel (150) is prevented.

68. The tissue treatment device (102) of claim 66, wherein, when the tissue treatment device (102) is in the irrigation mode or the suction mode, the ablation electrode (116) is retracted within the electrode channel.

69. The tissue treatment device (102) of claim 66, wherein the trigger assembly (136) comprises
a trigger (310) configured to be selectively manipulated by a user into one of an irrigation trigger position, a suction trigger position, and an ablation trigger position,
a first linkage (350) mechanically coupled between the trigger (310) and the fluid manifold (133), wherein the first linkage (350) is configured to set the fluid manifold (133) into the irrigation state in response to manipulation of the trigger (310) into the irrigation trigger position, thereby placing the tissue treatment device (102) in the irrigation mode, and wherein the first linkage (350) is further configured to set the fluid manifold (133) into the suction state in response to manipulation of th0 trigger (310) into the suction trigger position, thereby placing the tissue treatment device (102) in the suction mode, and
a second linkage (361, 362) mechanically coupled between the trigger and the electrode drive carriage (360), wherein the second linkage (361, 362) is configured to set the electrode drive carriage (360) into the electrode extension state in response to manipulation of the trigger (310) into the ablation trigger position, thereby placing the tissue treatment device (102) in the ablation mode.

70. The tissue treatment device of claim 69,
wherein the fluid manifold (133) comprises a stationary body (332) affixed to the handle body (130), and a movable body (334) that is rotatable about a pivot axis relative to the stationary body, wherein the handle body (130) and movable body (334) define the fluid manifold chamber (320) therebetween, and wherein the stationary body (332) comprises each of the irrigation port (322), the suction port (324), and the common port (326), and wherein the movable body (334) comprises an irrigation stopper (342) and a suction stopper (344), and
wherein the first linkage (350) is affixed to and configured to move the movable body (334) relative to the stationary body (332) in response to manipulation of the trigger (310) into the irrigation trigger position in which the irrigation stopper (342) does not cover the irrigation port (322) while the suction stopper (344) covers the suction port (324), thereby setting the fluid manifold (133) in the irrigation state, and wherein the first linkage (350) is further configured to move the movable body (334) relative to the stationary body (332) in response to manipulation of the trigger (310) into the suction trigger position in which the irrigation stopper (342) covers the irrigation port (322) while the suction stopper (344) does not cover the suction port (324), thereby setting the fluid manifold (133) in the suction state.

71. The tissue treatment device of claim 70,
wherein, when the tissue treatment device (102) is in the ablation mode, fluid communication between the suction tube (118B) and the irrigation/suction channel (150) of the elongate shaft (112) is allowed, while fluid communication between the irrigation tube (118A) and the irrigation/suction channel (150) is prevented, and
wherein the first linkage (350) is configured to set the fluid manifold (133) into the suction state in response to manipulation of the trigger (310) into the ablation trigger position, thereby placing the tissue treatment device (102) into the ablation mode.

72. The tissue treatment device of claim 71,
wherein, when the tissue treatment device (102) is in the irrigation mode or the suction mode, the ablation electrode (116) is retracted within the electrode channel, and
wherein the second linkage (361, 362) is configured to set the electrode drive carriage (360) into the electrode retraction state in response to manipulation of trigger (310) into either of the irrigation trigger position and the suction trigger position, thereby placing the tissue treatment device (102) into one of the irrigation mode and the suction mode.

73. The tissue treatment device of claim 72,
wherein the trigger assembly (136) is configured to selectively set the tissue treatment device (102) in a resting mode in which fluid communication between the irrigation tube (118A) and the irrigation/suction channel (150) of the elongate shaft (112) is prevented, fluid communication between the suction tube (118B) and the irrigation/suction channel (150) of the elongated shaft (112) is prevented, and the ablation electrode (116) is retracted within the electrode channel,
wherein the fluid manifold (133) is configured to be set in one of the irrigation state, the suction state, and a no-irrigation/no suction-state, wherein, in the no-irrigation/no suction-state, the irrigation port (322) is not fluidly coupled to the common port (326) via the chamber (320), and the suction port (324) is not fluidly coupled to the common port (326) via the chamber (320),
wherein the trigger (310) is configured to be selectively manipulated into one of the irrigation trigger position, the suction trigger position, the ablation trigger position, and a release trigger position, and
wherein the first linkage (350) is configured to set the fluid manifold (133) into the no-irrigation state/no-suction state, and the second linkage (361, 362) is configured to set the electrode drive carriage (360) into the electrode retraction state, respectively, in response to manipulation of the trigger (310) into the release trigger position to thereby place the tissue treatment device (102) into the resting mode.

74. The tissue treatment device (102) of claim 66, wherein the trigger assembly (136) is configured to directly transition the tissue treatment device (102) from the irrigation mode to the suction mode to thereby directly transition the tissue treatment device (102) from the suction mode to the ablation mode.

75. The tissue treatment device (102) of claim 74, wherein the trigger assembly (136) comprises a trigger (310) configured to be moved along a single direction to transition the tissue treatment device (102) from the irrigation mode to the suction mode, and then from the suction mode to the ablation mode.

76. The tissue treatment device (102) of claim 66, wherein the elongate shaft (112) is shaped and sized for insertion into a human urethra.

77. A tissue treatment device (102), comprising:
an elongate shaft (112) having a proximal end portion, a distal end portion, an irrigation/suction channel (150) extending between the proximal end portion and the distal end portion, and an electrode channel extending between the proximal end portion and the distal end portion;
an ablation electrode (116) slidably disposed within the electrode channel of the elongate shaft;
an irrigation tube (118A);
a suction tube (118B); and
a handle assembly (110) disposed at the proximal end portion of the elongate shaft, the handle assembly comprising
a handle body (130) at least partially receiving the suction tube and the irrigation tube; and
a trigger assembly (136) configured to selectively set the tissue treatment device in one of an irrigation mode, a suction mode, and an ablation mode, wherein in the irrigation mode, fluid communication between the irrigation tube (118A) and the irrigation/suction channel (150) of the elongate shaft (112) is allowed, while fluid communication between the suction tube (118B) and the irrigation/suction channel (150) is prevented, in the suction mode, fluid communication between the suction tube (118B) and the irrigation/suction channel (150) of the elongate shaft (112) is allowed, while fluid communication between the irrigation tube (118A) and the irrigation/suction channel (150) is prevented, and in the ablation mode, the ablation electrode (116) extends from an end of the electrode channel;
characterized in that the handle assembly further comprises
a fluid manifold (133), the fluid manifold having a chamber (320), an irrigation port (322) fluidly coupled to the irrigation tube (118A), a suction port (324) fluidly coupled to the suction tube (118B), and a common port (326) fluidly coupled to the irrigation/suction channel (150) of elongate shaft (112), wherein the fluid manifold (133) is configured to be set in one of an irrigation state, wherein the irrigation port (322) is fluidly coupled to the common port (326) via the chamber (320), and the suction port (324) is not fluidly coupled to the common port (326) via the chamber (320), and a suction state, wherein the suction port (324) is fluidly coupled to the common port (326) via the chamber (320), and the irrigation port (322) is not fluidly coupled to the common port (326) via the chamber (320), and
an electrode drive carriage (360) to which the ablation electrode (116) is affixed, wherein the electrode drive carriage (360) is configured to be set in an electrode extension state, wherein the ablation electrode (116) extends from the electrode channel to place the tissue treatment device in the ablation mode, and an electrode retraction state,
wherein, when the tissue treatment device (102) is in the ablation mode, fluid communication between the suction tube (118B) and the irrigation/suction channel (150) of the elongate shaft (112) is allowed, while fluid communication between the irrigation tube (118A) and the irrigation/suction channel (150) is prevented, and
wherein, when the tissue treatment device (102) is in the irrigation mode or the suction mode, the ablation electrode (116) is retracted within the electrode channel.
